SEQUENCE LISTING (18 sequences)

SEQ ID NO: 1 - DNA, 23 bp, Artificial Sequence
  Human PCK1 sgRNA 1
gaataagccagatgtaatcaggg

SEQ ID NO: 2 - DNA, 23 bp, Artificial Sequence
  Human PCK1 sgRNA 2
actcaggccctgattacatctgg

SEQ ID NO: 3 - DNA, 23 bp, Artificial Sequence
  Human PCK1 sgRNA 3
tctctctctcgatttcacagggg

SEQ ID NO: 4 - DNA, 7024 bp, Artificial Sequence
  hPCK1 2A-mSc-2A-Luc
  misc_feature (1)..(1561): 5' PCK1 homology
  misc_feature (1565)..(1630): P2A self-cleaving peptide
  misc_feature (1631)..(2326): mScarlet
  misc_feature (2327)..(2389): T2A self-cleaving peptide
  misc_feature (2390)..(4042): Luciferase
  misc_feature (4043)..(4076): LoxP
  misc_feature (4077)..(5319): NeoR
  misc_feature (5320)..(5353): LoxP
  misc_feature (5379)..(7024): 3' PCK1 homology
ccaatgccatcaagaccatccagaagaacacaatctttaccaatgtggccgagaccagcgacgggggcgtttactgggaaggcattgatgagccgctagcttcaggtgtcaccatcacgtcctggaagaataaggagtggagctcagaggatggtgtgtccctgccagaggccttggtgtgccgggctgcagggactgcctgtttgagccaggcactcacgagcctttctctgtcttataggggaaccttgtgcccaccccaactcgaggttctgcacccctgccagccagtgccccatcattgatgctgcctgggagtctccggaaggtgttcccattgaaggcattatctttggaggccgtagacctgctggtgaggctctccttcatttaggctgggaacatgggtgtgctgggtaccgaagggcatctgtgaaatctctccttttccatgaccttgtcagagggtgcccaggggcttcctttcttgagcttccttcccaaagatccagaataattggcaagttcaaatgtagaaccaacccttctggtcacctggaacctttctgaatccctgatctattgtagcttgatcaaattttactttttactttgtggcctcagtcatgtaactttgagttagcagttttctgcaatttagcttggtgaacgcaaaactagctcgattacaaagttattgtcttgccgtgtctttccgtgttgtgaataacaccactggttgtggagtctgaatttcaaagcctctgatgaacatttctctttttttttcctgctaaaggtgtccctctagtctatgaagctctcagctggcaacatggagtctttgtgggggcggccatgagatcagaggccacagcggctgcagaacataaaggtaaatcaaagtcctgatctgaaaccacagagaagtgggattagagcactcttcgtcactcttatgtctctctccttttctgtgtctgtgtgtggggagagagagagagagaaagagagagaggagaacaaagcatgctaatgtcaacaatcaatggcgtcagtcttgcctaggagagcctcatttactaatgaactccctctctgtttaacaggcaaaatcatcatgcatgacccctttgccatgcggcccttctttggctacaacttcggcaaatacctggcccactggcttagcatggcccagcacccagcagccaaactgcccaagatcttccatgtcaactggttccggaaggacaaggaaggcaaattcctctggccaggctttggagagaactccagggtgctggagtggatgttcaaccggatcgatggaaaagccagcaccaagctcacgcccataggctacatccccaaggaggatgccctgaacctgaaaggcctggggcacatcaacatgatggagcttttcagcatctccaaggaattctgggagaaggaggtggaagacatcgagaagtatctggaggatcaagtcaatgccgacctcccctgtgaaatcgagagagagatccttgccttgaagcaaagaataagccagatggggggatccggagctactaacttcagcctgctgaagcaggctggagatgtggaggagaaccctggacctatggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgacaaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctgcgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcgggcgctacctggcggacttcaagaccacctacaaggccaagaagcccgtgcagatgcccggcgcctacaacgtggaccgcaagttggacatcacctcccacaacgaggactacaccgtggtggaacagtacgaacgctccgagggccgccactccaccggcggcatggacgagctgtacaagggcagcggagagggcagaggaagtcttctaacatgcggtgacgtggaggagaatcccggccctatggaagacgccaaaaacataaagaaaggcccggcgccattctatccgctagaggatggaaccgctggagagcaactgcataaggctatgaagagatacgccctggttcctggaaccattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatctataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacaccttcgtcacatctcatctacctcccggttttaatgaatacgattttgtaccagagtcctttgatcgtgacaaaacaattgcactgataatgaactcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaggagctgtttttacgatcccttcaggattacaaaatccaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggataccaggtggcccccgctgaattggagtcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgccaaaaagttgcgcggaggagttgtgtttgtggacgaagtaccgaaaggtcttaccggaaaactcgacgcaagaaaaatcagagagatcctcataaaggccaagaagggcggaaagtccaaattgtaaataacttcgtatagcatacattatacgaagttatatgatatcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagggtaccgccaccatggggtaccgccaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtcgacacgcgttggtaccataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctataacttcgtatagcatacattatacgaagttatgacagattgcctttcctttaggcaggtaatcagggcctgagtgctttacctttaaaatcattccctttcccatccataaggtgcagtaggagcaagagagggcaagtgttcccaaattgacgccaccataataatcatcaccacaccgtgagcagatctgaaaggcacactttgatttttttaaggataagaaccacagaacactgggtagtagctaatgaaattgagaagggaaatcttagcatgcctccaaaaattcacatccaatgcatagtttgttcaaatttaaggttactcaggcattgatcttttcagtgttttttcactttagctatgtggattagctagaatgcacaccaaaaaaatacttgagctgtatatatatatgtgtgtgtgtgtgtgtgtgtgtgtgtgtgtgtgtgcatgtatgtgcacatgtgtctgtgtggtatatttgtgtatgtgtatttgtatgtactgttattgaaaatatatttaatacctttggaaaaatcttgggcaagatgacctactagttttccttgaaaaaaagttgctttgttattaatattgtgcttaaattatttttatacaccattgttccttacctttacataattgcaatatttcccccttactacttcttggaaaaaaattacaaaatgaagttttatagaaaagatggatttgctttgcttggtttttcttatttgagctaccaagaggagagaactctgattaaatatcttgtgatttaaatcctgccccctccccaagaaggtggggacgttcagaatcacacttgaagagggggtgcattgcctggtcccagggagaaggagggagcagatggggaggaatgaggcagggaaccatggttggggttttctgaatttgctgaagccactagttggcatcctttaacaattctacaaacctttagatggtgaggcttatgtggcaggactgggagggtttaatgcatgctttttgacagtttggatctggaggggtctcaggcttccacaaaaattacattgtggacagtttcataggattttgccttttaactcttcaaagcaacagttttctcaactgtcatccccgcgggggagcagaggaagacaagtgagaaggaaaaggggacaggatctgggttgagcaagcattaattgagcgccttctgtatcccggacatcatgccaacaccatgggctgcccgagaagagaggtgggtggtttggtccccagctctgccttgaaccctcacctgctctcctctactcccagtgtttattggatattccagaacagtccaaacgaaccataaactttatatcacagattcaatctgtgtaaatactattatcttcaagttgtagtttctttcttgggcccaggctgtgaaattggcttcacccctaccacaccccagcgggcaggcttgttcttatgtcacactccacccctccagtctcctcaactggaaaacttgcctcttttcctcacttcctctgctccttacaatggcttcatatggaaggtgaagctgagatcaccccaattttacaggtgaggaaactgaggtcaagctaggagcgtccagcggggctgagaaattgccaggatttccttccccattaaggagctgagagtcttagctccagacac

SEQ ID NO: 5 - protein, 825 aa, Artificial Sequence
  P2A-mScarlet-T2A-Luciferase
GSGATNFSLLKQAGDVEENPGPMVSKGEAVIKEFMRFKVHMEGSMNGHEFEIEGEGEGRPYEGTQTAKLKVTKGGPLPFSWDILSPQFMYGSRAFIKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYKGSGEGRGSLLTCGDVEENPGPMEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 6 - DNA, 1561 bp, Homo sapiens
ccaatgccatcaagaccatccagaagaacacaatctttaccaatgtggccgagaccagcgacgggggcgtttactgggaaggcattgatgagccgctagcttcaggtgtcaccatcacgtcctggaagaataaggagtggagctcagaggatggtgtgtccctgccagaggccttggtgtgccgggctgcagggactgcctgtttgagccaggcactcacgagcctttctctgtcttataggggaaccttgtgcccaccccaactcgaggttctgcacccctgccagccagtgccccatcattgatgctgcctgggagtctccggaaggtgttcccattgaaggcattatctttggaggccgtagacctgctggtgaggctctccttcatttaggctgggaacatgggtgtgctgggtaccgaagggcatctgtgaaatctctccttttccatgaccttgtcagagggtgcccaggggcttcctttcttgagcttccttcccaaagatccagaataattggcaagttcaaatgtagaaccaacccttctggtcacctggaacctttctgaatccctgatctattgtagcttgatcaaattttactttttactttgtggcctcagtcatgtaactttgagttagcagttttctgcaatttagcttggtgaacgcaaaactagctcgattacaaagttattgtcttgccgtgtctttccgtgttgtgaataacaccactggttgtggagtctgaatttcaaagcctctgatgaacatttctctttttttttcctgctaaaggtgtccctctagtctatgaagctctcagctggcaacatggagtctttgtgggggcggccatgagatcagaggccacagcggctgcagaacataaaggtaaatcaaagtcctgatctgaaaccacagagaagtgggattagagcactcttcgtcactcttatgtctctctccttttctgtgtctgtgtgtggggagagagagagagagaaagagagagaggagaacaaagcatgctaatgtcaacaatcaatggcgtcagtcttgcctaggagagcctcatttactaatgaactccctctctgtttaacaggcaaaatcatcatgcatgacccctttgccatgcggcccttctttggctacaacttcggcaaatacctggcccactggcttagcatggcccagcacccagcagccaaactgcccaagatcttccatgtcaactggttccggaaggacaaggaaggcaaattcctctggccaggctttggagagaactccagggtgctggagtggatgttcaaccggatcgatggaaaagccagcaccaagctcacgcccataggctacatccccaaggaggatgccctgaacctgaaaggcctggggcacatcaacatgatggagcttttcagcatctccaaggaattctgggagaaggaggtggaagacatcgagaagtatctggaggatcaagtcaatgccgacctcccctgtgaaatcgagagagagatccttgccttgaagcaaagaataagccagatg

SEQ ID NO: 7 - DNA, 66 bp, Artificial Sequence
  P2A nucleic acid
ggatccggagctactaacttcagcctgctgaagcaggctggagatgtggaggagaaccctggacct

SEQ ID NO: 8 - DNA, 696 bp, Artificial Sequence
  mScarlet nucleic acid
atggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgacaaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctgcgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcgggcgctacctggcggacttcaagaccacctacaaggccaagaagcccgtgcagatgcccggcgcctacaacgtggaccgcaagttggacatcacctcccacaacgaggactacaccgtggtggaacagtacgaacgctccgagggccgccactccaccggcggcatggacgagctgtacaag

SEQ ID NO: 9 - DNA, 63 bp, Artificial Sequence
  T2A nucleic acid
ggcagcggagagggcagaggaagtcttctaacatgcggtgacgtggaggagaatcccggccct

SEQ ID NO: 10 - DNA, 1653 bp, Artificial Sequence
  Luciferase nucleic acid
atggaagacgccaaaaacataaagaaaggcccggcgccattctatccgctagaggatggaaccgctggagagcaactgcataaggctatgaagagatacgccctggttcctggaaccattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatctataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacaccttcgtcacatctcatctacctcccggttttaatgaatacgattttgtaccagagtcctttgatcgtgacaaaacaattgcactgataatgaactcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaggagctgtttttacgatcccttcaggattacaaaatccaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggataccaggtggcccccgctgaattggagtcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgccaaaaagttgcgcggaggagttgtgtttgtggacgaagtaccgaaaggtcttaccggaaaactcgacgcaagaaaaatcagagagatcctcataaaggccaagaagggcggaaagtccaaattgtaa

SEQ ID NO: 11 - DNA, 1243 bp, Artificial Sequence
  NeoR cassette
atgatatcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagggtaccgccaccatggggtaccgccaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtcgacacgcgttggtaccataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctct

SEQ ID NO: 12 - DNA, 34 bp, Artificial Sequence
  LoxP nucleic acid
ataacttcgtatagcatacattatacgaagttat

SEQ ID NO: 13 - DNA, 1646 bp, Homo sapiens
gtaatcagggcctgagtgctttacctttaaaatcattccctttcccatccataaggtgcagtaggagcaagagagggcaagtgttcccaaattgacgccaccataataatcatcaccacaccgtgagcagatctgaaaggcacactttgatttttttaaggataagaaccacagaacactgggtagtagctaatgaaattgagaagggaaatcttagcatgcctccaaaaattcacatccaatgcatagtttgttcaaatttaaggttactcaggcattgatcttttcagtgttttttcactttagctatgtggattagctagaatgcacaccaaaaaaatacttgagctgtatatatatatgtgtgtgtgtgtgtgtgtgtgtgtgtgtgtgtgtgcatgtatgtgcacatgtgtctgtgtggtatatttgtgtatgtgtatttgtatgtactgttattgaaaatatatttaatacctttggaaaaatcttgggcaagatgacctactagttttccttgaaaaaaagttgctttgttattaatattgtgcttaaattatttttatacaccattgttccttacctttacataattgcaatatttcccccttactacttcttggaaaaaaattacaaaatgaagttttatagaaaagatggatttgctttgcttggtttttcttatttgagctaccaagaggagagaactctgattaaatatcttgtgatttaaatcctgccccctccccaagaaggtggggacgttcagaatcacacttgaagagggggtgcattgcctggtcccagggagaaggagggagcagatggggaggaatgaggcagggaaccatggttggggttttctgaatttgctgaagccactagttggcatcctttaacaattctacaaacctttagatggtgaggcttatgtggcaggactgggagggtttaatgcatgctttttgacagtttggatctggaggggtctcaggcttccacaaaaattacattgtggacagtttcataggattttgccttttaactcttcaaagcaacagttttctcaactgtcatccccgcgggggagcagaggaagacaagtgagaaggaaaaggggacaggatctgggttgagcaagcattaattgagcgccttctgtatcccggacatcatgccaacaccatgggctgcccgagaagagaggtgggtggtttggtccccagctctgccttgaaccctcacctgctctcctctactcccagtgtttattggatattccagaacagtccaaacgaaccataaactttatatcacagattcaatctgtgtaaatactattatcttcaagttgtagtttctttcttgggcccaggctgtgaaattggcttcacccctaccacaccccagcgggcaggcttgttcttatgtcacactccacccctccagtctcctcaactggaaaacttgcctcttttcctcacttcctctgctccttacaatggcttcatatggaaggtgaagctgagatcaccccaattttacaggtgaggaaactgaggtcaagctaggagcgtccagcggggctgagaaattgccaggatttccttccccattaaggagctgagagtcttagctccagacac

SEQ ID NO: 14 - protein, 22 aa, Artificial Sequence
  P2A peptide
GSGATNFSLLKQAGDVEENPGP

SEQ ID NO: 15 - protein, 232 aa, Artificial Sequence
  mScarlet peptide
MVSKGEAVIKEFMRFKVHMEGSMNGHEFEIEGEGEGRPYEGTQTAKLKVTKGGPLPFSWDILSPQFMYGSRAFIKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYK

SEQ ID NO: 16 - protein, 21 aa, Artificial Sequence
  T2A peptide
GSGEGRGSLLTCGDVEENPGP

SEQ ID NO: 17 - protein, 550 aa, Artificial Sequence
  Luciferase peptide
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 18 - protein, 267 aa, Artificial Sequence
  NeoR peptide
MGSAIEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKARMPDGDDLVVTHGDACLPNIMVENGRFSGFIDCGRLGVADRYQDIALATRDIAEELGGEWADRFLVLYGIAAPDSQRIAFYRLLDEFF

(19) United States
(12) Patent Application Publication
(10) Pub. No.: US 2023/0235316 A1
(43) Pub. Date: Jul. 27, 2023

(54) MODEL FOR INSULIN RESISTANCE

(71) Applicant: CHILDREN'S HOSPITAL MEDICAL CENTER, Cincinnati, OH (US)

(72) Inventors: Takanori Takebe, Cincinnati, OH (US); Masaki Kimura, Cincinnati, OH (US)

(21) Appl. No.: 18/003,130

(22) PCT Filed: Jun. 22, 2021
(86) PCT No.: PCT/US2021/038416

§ 371 Date: Dec. 22, 2022

Related U.S. Application Data
(60) Provisional application No. 63/042,997, filed on Jun. 23, 2020.

(51) Int. Cl. C12N 15/10; G01N 33/50

(52) U.S. Cl. CPC C12N 15/1055; G01N 33/5082

(57) ABSTRACT

Disclosed herein are insulin resistance reporters for use in quantifying insulin response in biological cells. These biological cells may be stem cell compositions or derivatives thereof comprising the insulin resistance reporter. The stem cell derivatives include but are not limited to insulin responsive cells, tissues, or organoids, such as pancreatic, brain, adipose, muscle, or liver cells, or tissues or organoids thereof. Also disclosed herein are methods of using said insulin resistance reporters and cells with these insulin resistance reporters as models to examine insulin resistance and screening for compounds that are potentially useful for the treatment of diseases or disorders associated with insulin resistance. The cells comprising an insulin resistance reporter may be hepatic cells or liver organoid compositions, which can be used in investigating hepatic insulin resistance, for example, as a result of non-alcoholic fatty liver disease or steatohepatitis.

### CROSS-REFERENCE TO RELATED APPLICATIONS

This application claims the benefit of priority to U.S. Provisional Pat. Application No. 63/042,997, filed Jun. 23, 2020, which is hereby expressly incorporated by reference in its entirety.

### STATEMENT REGARDING FEDERALLY SPONSORED R&D

This invention was made with government support under DK119982 awarded by the National Institutes of Health. The government has certain rights to the invention.

### REFERENCE TO SEQUENCE LISTING

The present application is being filed along with a Sequence Listing in electronic format. The Sequence Listing is provided as a file entitled SeqListingCHMC63_032WO.TXT, which was created and last modified on Jun. 21, 2021, which is 38,040 bytes in size. The information in the electronic Sequence Listing is hereby incorporated by reference in its entirety.

### FIELD OF THE INVENTION

Aspects of the present disclosure relate generally to insulin response reporters. These insulin response reporters may be used in cells, such as stem cells, or derivatives of the cells, such as differentiated cells or organoids, for example, for detecting an insulin response, or presence or lack thereof, or for screening for compounds that affect insulin response in cells. The present disclosure also describes methods of engineering the insulin response reporters and making and using cells with the insulin response reporters.

### BACKGROUND

Nonalcoholic fatty liver disease (NALFD) and associated metabolic syndromes continue to be on the rise in the human population, leading to increased morbidity and mortality. Hepatic insulin resistance is oftentimes accompanied by NAFLD, which is a risk factor for co-morbidities such as heart disease. In contract to systemic insulin resistance, NAFLD can cause hepatic insulin resistance through increased hepatic fat accumulation, a detrimental process that is highly variable and dependent on heritable and non-heritable factors. However, the precise genetic and molecule mechanisms linking hepatic fat accumulation and insulin resistance is not fully understood and confounded by lack of suitable human-based model systems. Therefore, there is a present need for robust and reliable models to study a wide range of liver disorders such as hepatic insulin resistance. Furthermore, there is a need for improved screening methods using human-based model systems for discovery of potential treatments for insulin resistance.

### SUMMARY

Disclosed herein are liver organoids comprising an insulin resistance reporter. In some embodiments, the insulin resistance reporter is operatively linked to an insulin-dependent gene of the liver organoid. An insulin-dependent gene may be any gene (and resultant protein) that is modulated by an effect of insulin or an insulin signaling pathway that occurs in biological cells. In some embodiments, the insulin-dependent gene is a gluconeogenesis gene or a lipogenesis gene. The insulin resistance reporter may be any one of the insulin resistance reporters disclosed herein.

Also disclosed herein are insulin responsive cells, tissues, or organoids comprising an insulin resistance reporter. In some embodiments, the insulin resistance reporter is operatively linked to an insulin-dependent gene of the insulin responsive cell, tissue, or organoid. The insulin responsive cells, tissues, or organoids may be or may comprise any one or more cell types that undergo a functional response to insulin or an insulin signaling pathway. In some embodiments, the insulin responsive cells, tissues, or organoids may be affected by a disease or disorder associated with a dysfunctional insulin response, such as insulin resistance. Expressing an insulin resistance reporter in the insulin responsive cells, tissues, or organoids may be useful in detecting the disease or disorder associated with the dysfunctional insulin response, or identifying molecules or compounds that are useful in treating the disease or disorder.

Also disclosed herein are insulin resistance reporters. In some embodiments, the insulin resistance reporters comprise one or more reporter genes flanked by a 5′ homology region and a 3′ homology region associated with an insulin-dependent gene. In some embodiments, these insulin resistance reporters are nucleic acids. In some embodiments, the insulin resistance reporters are intended to be integrated to the genome at the insulin-dependent gene, for example, through homologous recombination through the 5′ homology region and 3′ homology region associated with the insulin-dependent gene. In some embodiments, the insulin resistance reporters are integrated into an insulin responsive cell, tissue, or organoid. In some embodiments, the insulin resistance reporters are integrated into a liver organoid, such as a fatty liver organoid or steatohepatitis liver organoid.

Also disclosed herein are in vitro methods of screening for candidate compounds for the treatment of a disease or disorder associated with insulin dysfunction. In some embodiments, the methods comprise contacting a liver organoid comprising an insulin resistance reporter or an insulin responsive cell, tissue, or organoid comprising an insulin resistance reporter with the candidate compounds, and observing an improvement in the disease or disorder associated with insulin dysfunction in the liver organoid or the insulin responsive cell, tissue, or organoid.

Also disclosed herein are stem cells comprising an insulin resistance reporter. In some embodiments, the insulin resistance reporter is any one of the insulin resistance reporters disclosed herein. Also disclosed herein definitive endoderm cells differentiated from any one of the stem cells disclosed herein. Also disclosed herein are anterior foregut cells differentiated from any one of the stem cells disclosed herein. Also disclosed herein are insulin responsive cells, tissues, or organoids differentiated from any one of the stem cells disclosed herein. Also disclosed herein are pancreatic cells, brain cells, adipose cells, muscle cells, or liver cells differentiated from any one of the stem cells disclosed herein. Also disclosed herein are liver organoids differentiated from any one of the stem cells, definitive endoderm, or anterior foregut cells disclosed herein.

Also disclosed herein are in vitro methods of assessing insulin resistance of an insulin responsive cell, tissue, or organoid comprising an insulin resistance reporter. In some embodiments, the methods comprise quantifying a baseline expression level of one or more reporter proteins of the insulin resistance reporter, contacting the insulin responsive cell, tissue, or organoid with insulin or a derivative or mimetic thereof, quantifying a post-treatment expression level of the one or more reporter proteins, and determining based on the change in expression level or lack thereof of the one or more reporter proteins that the insulin responsive cell, tissue, or organoid exhibits insulin resistance.

Also disclosed herein are in vitro methods of screening for a compound or composition that treats insulin resistance. In some embodiments, the methods comprise contacting an insulin responsive cell, tissue, or organoid comprising an insulin resistance reporter with one or more fatty acids, quantifying a baseline expression level of one or more reporter proteins of the insulin resistance reporter, contacting the insulin responsive cell, tissue, or organoid with the compound or composition, quantifying a post-treatment expression level of the one or more reporter proteins, and determining based on the change in expression level or lack thereof of the one or more reporter proteins that the compound or composition can treat the insulin resistance.

Also disclosed herein are the compounds or compositions identified by any one of the screening methods disclosed herein. Also disclosed herein are pharmaceutical compositions comprising any one or more of the compounds or compositions identified by any one of the screening methods disclosed herein. Also disclosed herein are methods of treating insulin resistance in a subject in need thereof by administering any one of the compounds or compositions identified to the subject.

Also disclosed herein are methods of monitoring insulin response in a subject. In some embodiments, the methods comprise transplanting a liver organoid comprising an insulin resistance reporter or an insulin responsive cell, tissue, or organoid comprising an insulin resistance reporter to the subject and monitoring expression of the insulin resistance reporter of the liver organoid or the insulin responsive cell, tissue, or organoid.

Exemplary embodiments of the present disclosure are provided in the following numbered embodiments:

1. A liver organoid comprising an insulin resistance reporter.

2. The liver organoid of any one of the preceding embodiments, wherein the liver organoid is a human liver organoid (HLO).

3. The liver organoid of any one of the preceding embodiments, wherein the liver organoid is produced from induced pluripotent stem cells (iPSCs).

4. The liver organoid of any one of the preceding embodiments, wherein the liver organoid is produced from iPSCs derived from a human subject.

5. The liver organoid of embodiment 3 or 4, wherein the iPSCs are first differentiated into anterior foregut cells.

6. The liver organoid of embodiment 5, wherein the anterior foregut cells are cryopreserved for a period of time and thawed before differentiating the anterior foregut cells into the liver organoid.

7. The liver organoid of any one of the preceding embodiments, wherein the insulin resistance reporter comprises one or more nucleic acid sequences encoding for a reporter protein and one or more nucleic acid sequences encoding for a self-cleaving peptide separating each of the one or more nucleic acid sequence encoding for the reporter protein.

8. The liver organoid of any one of the preceding embodiments, wherein the insulin resistance reporter comprises one or more nucleic acid sequences having at least 80%, 85%, 90%, 91%, 92%, 93%, 94%, 95%, 96%, 97%, 98%, or 99% homology to a sequence encoding for a reporter protein and one or more nucleic acid sequences having at least 80%, 85%, 90%, 91%, 92%, 93%, 94%, 95%, 96%, 97%, 98%, or 99% homology to a sequence encoding for a self-cleaving peptide separating each of the sequences encoding for the reporter protein.

9. The liver organoid of any one of the preceding embodiments, wherein the reporter protein is a fluorescent protein.

10. The liver organoid of any one of the preceding embodiments, wherein the fluorescent protein is mScarlet.

11. The liver organoid of any one of the preceding embodiments, wherein the reporter protein is a luminescent protein.

12. The liver organoid of any one of the preceding embodiments, wherein the luminescent protein is luciferase.

13. The liver organoid of any one of the preceding embodiments, wherein the insulin resistance reporter further comprises one or more nucleic acid sequences having at least 80%, 85%, 90%, 91%, 92%, 93%, 94%, 95%, 96%, 97%, 98%, 99%, or 100% homology to a sequence associated with an insulin-dependent gene.

14. The liver organoid of any one of the preceding embodiments, wherein the insulin-dependent gene is a gluconeogenesis gene or a lipogenesis gene.

15. The liver organoid of any one of the preceding embodiments, wherein the insulin-dependent gene is PCK1.

16. The liver organoid of any one of the preceding embodiments, wherein the insulin-dependent gene is selected from the group consisting of PCK1, G6PC, G6PC2, G6PC3, GSK3A, GSK3B, MTOR, GCK, FOXO1, CREB1, TFE1, TFE3, SREBP1C, FASN, ACLY, and ACC.

17. The liver organoid of any one of the preceding embodiments, wherein the one or more nucleic acid sequences having homology to the sequence associated with the insulin-dependent gene are flanking the sequences encoding for the reporter protein and the sequences encoding for the self-cleaving peptide, wherein the sequences having homology to the sequence associated with the insulin-dependent gene act as homology regions for recombination into the genome of the liver organoid.

18. The liver organoid of any one of the preceding embodiments, wherein the insulin resistance reporter has been integrated into the genome of the liver organoid using a CRISPR nuclease.

19. The liver organoid of any one of the preceding embodiments, wherein the CRISPR nuclease is Cas9.

20. The liver organoid of any one of the preceding embodiments, wherein the liver organoid is a fatty liver organoid after treatment of the liver organoid with one or more fatty acids, and wherein the fatty liver organoid exhibits insulin resistance.

21. The liver organoid of any one of the preceding embodiments, wherein the one or more fatty acids comprise oleic acid, linoleic acid, palmitic acid, or any combination thereof.

22. The insulin resistance reporter of any one of the preceding embodiments.

23. An in vitro method of assessing hepatic insulin resistance of a liver organoid comprising an insulin resistance reporter, comprising:
quantifying a baseline expression level of one or more reporter proteins of the insulin resistance reporter;
contacting the liver organoid with insulin or a derivative or mimetic thereof;
quantifying a post-treatment expression level of the one or more reporter proteins; and
determining based on the change in expression level or lack thereof of the one or more reporter proteins that the liver organoid exhibits hepatic insulin resistance.

24. The method of embodiment 23, further comprising contacting the liver organoid with one or more of obeticholic acid (OCA), pioglitazone, or metformin, or any combination thereof.

25. The method of embodiment 23 or 24, further comprising contacting the liver organoid with one or more fatty acids prior to quantifying the baseline expression level.

26. The method of any one of embodiments 23-25, further comprising contacting the liver organoid with one or more fatty acids after quantifying the baseline expression level and prior to contacting the liver organoid with insulin or the derivative or mimetic thereof.

27. An in vitro method of screening for a compound or composition that treats hepatic insulin resistance caused by nonalcoholic fatty liver disease (NAFLD), comprising:
contacting a liver organoid comprising an insulin resistance reporter with one or more fatty acids;
quantifying a baseline expression level of one or more reporter proteins of the insulin resistance reporter;
contacting the liver organoid with the compound or composition;
quantifying a post-treatment expression level of the one or more reporter proteins; and
determining based on the change in expression level or lack thereof of the one or more reporter proteins that the compound or composition can treat the hepatic insulin resistance.

28. The method of any one of embodiments 23-27, wherein the liver organoid is the liver organoid of any one of embodiments 1-21.

29. The method of any one of embodiments 23-28, wherein the insulin resistance reporter is the insulin resistance reporter of embodiment 22.

30. The method of any one of embodiments 23-29, wherein the one or more fatty acids comprise oleic acid, linoleic acid, palmitic acid, or any combination thereof.

31. The method of any one of embodiments 23-30, wherein the liver organoid is derived from a human subject in need of treatment for hepatic insulin resistance.

32. A stem cell comprising an insulin resistance reporter.

33. The stem cell of any one of the preceding embodiments, wherein the stem cell is an induced pluripotent stem cell (iPSC).

34. The stem cell of any one of the preceding embodiments, wherein the stem cell is derived from a human subject.

35. The stem cell of any one of the preceding embodiments, wherein the insulin resistance reporter comprises one or more nucleic acid sequences encoding for a reporter protein and one or more nucleic acid sequences encoding for a self-cleaving peptide separating each of the one or more nucleic acid sequence encoding for the reporter protein.

36. The stem cell of any one of the preceding embodiments, wherein the insulin resistance reporter comprises one or more nucleic acid sequences having at least 80%, 85%, 90%, 91%, 92%, 93%, 94%, 95%, 96%, 97%, 98%, or 99% homology to a sequence encoding for a reporter protein and one or more nucleic acid sequences having at least 80%, 85%, 90%, 91%, 92%, 93%, 94%, 95%, 96%, 97%, 98%, or 99% homology to a sequence encoding for a self-cleaving peptide separating each of the sequences encoding for the reporter protein.

37. The stem cell of any one of the preceding embodiments, wherein the reporter protein is a fluorescent protein.

38. The stem cell of any one of the preceding embodiments, wherein the fluorescent protein is mScarlet.

39. The stem cell of any one of the preceding embodiments, wherein the reporter protein is a luminescent protein.

40. The stem cell of any one of the preceding embodiments, wherein the luminescent protein is luciferase.

41. The stem cell of any one of the preceding embodiments, wherein the insulin resistance reporter further comprises one or more nucleic acid sequences having at least 80%, 85%, 90%, 91%, 92%, 93%, 94%, 95%, 96%, 97%, 98%, 99%, or 100% homology to a sequence associated with an insulin-dependent gene.

42. The stem cell of any one of the preceding embodiments, wherein the insulin-dependent gene is a gluconeogenesis gene or a lipogenesis gene.

43. The stem cell of any one of the preceding embodiments, wherein the insulin-dependent gene is PCK1.

44. The stem cell of any one of the preceding embodiments, wherein the insulin-dependent gene is selected from the group consisting of PCK1, G6PC, G6PC2, G6PC3, GSK3A, GSK3B, MTOR, GCK, FOXO1, CREB1, TFE1, TFE3, SREBP1C, FASN, ACLY, and ACC.

45. The stem cell of any one of the preceding embodiments, wherein the one or more nucleic acid sequences having homology to the sequence associated with the insulin-dependent gene are flanking the sequences encoding for the reporter protein and the sequences encoding for the self-cleaving peptide, wherein the sequences having homology to the sequence associated with the insulin-dependent gene act as homology regions for recombination into the genome of the liver organoid.

46. The stem cell of any one of the preceding embodiments, wherein the insulin resistance reporter has been integrated into the genome of the stem cell using a CRISPR nuclease.

47. The stem cell of any one of the preceding embodiments, wherein the CRISPR nuclease is Cas9.

48. A definitive endoderm cell differentiated from the stem cell of any one of embodiments 32-47.

49. An anterior foregut cell differentiated from the stem cell of any one of embodiments 32-47.

50. An insulin responsive cell, tissue, or organoid differentiated from the stem cell of any one of embodiments 32-47.

51. The insulin responsive cell, tissue, or organoid of embodiment 50, wherein the insulin responsive cell, tissue, or organoid comprises pancreatic cells, brain cells, adipose cells, muscle cells, or liver cells.

52. The insulin responsive cell, tissue, or organoid of embodiment 50 or 51, wherein the insulin responsive cell, tissue, or organoid is a liver organoid.

53. A pancreatic cell, brain cell, adipose cell, muscle cell, or liver cell differentiated from the stem cell of any one of embodiments 32-47.

54. A liver organoid differentiated from the stem cell of any one of embodiments 32-47, the definitive endoderm cell of embodiment 48, or the anterior foregut cell of embodiment 49, wherein the liver organoid comprises the insulin resistance reporter.

55. The liver organoid of embodiment 54, wherein the liver organoid is a fatty liver organoid after treatment of the liver organoid with one or more fatty acids, and wherein the fatty liver organoid exhibits insulin resistance.

56. The liver organoid of embodiment 55, wherein the one or more fatty acids comprise oleic acid, linoleic acid, palmitic acid, or any combination thereof.

57. The insulin resistance reporter of any one of the preceding embodiments.

58. An in vitro method of assessing insulin resistance of an insulin responsive cell, tissue, or organoid comprising an insulin resistance reporter, comprising:
quantifying a baseline expression level of one or more reporter proteins of the insulin resistance reporter;
contacting the insulin responsive cell, tissue, or organoid with insulin or a derivative or mimetic thereof;
quantifying a post-treatment expression level of the one or more reporter proteins; and
determining based on the change in expression level or lack thereof of the one or more reporter proteins that the insulin responsive cell, tissue, or organoid exhibits insulin resistance.

59. The method of embodiment 58, further comprising contacting the insulin responsive cell, tissue, or organoid with one or more of obeticholic acid (OCA), pioglitazone, or metformin, or any combination thereof.

60. The method of embodiment 58 or 59, further comprising contacting the insulin responsive cell, tissue, or organoid with one or more fatty acids prior to quantifying the baseline expression level.

61. The method of any one of embodiments 58-60, further comprising contacting the insulin responsive cell, tissue, or organoid with one or more fatty acids after quantifying the baseline expression level and prior to contacting the insulin responsive cell, tissue, or organoid with insulin or the derivative or mimetic thereof.

62. An in vitro method of screening for a compound or composition that treats insulin resistance, comprising:
contacting an insulin responsive cell, tissue, or organoid comprising an insulin resistance reporter with one or more fatty acids;
quantifying a baseline expression level of one or more reporter proteins of the insulin resistance reporter;
contacting the insulin responsive cell, tissue, or organoid with the compound or composition;
quantifying a post-treatment expression level of the one or more reporter proteins; and
determining based on the change in expression level or lack thereof of the one or more reporter proteins that the compound or composition can treat the insulin resistance.

63. The method of any one of embodiments 58-62, wherein the insulin responsive cell, tissue, or organoid is the insulin responsive cell, tissue, or organoid of any one of the preceding embodiments.

64. The method of any one of embodiments 58-63, wherein the insulin resistance reporter is the insulin resistance reporter of embodiment 26.

65. The method of any one of embodiments 58-64, wherein the one or more fatty acids comprise oleic acid, linoleic acid, palmitic acid, or any combination thereof.

66. The method of any one of embodiments 58-65, wherein the insulin responsive cell, tissue, or organoid is derived from a human subject in need of treatment for insulin resistance.

67. The method of any one of embodiments 58-66, wherein the insulin responsive cell, tissue, or organoid is a liver organoid and the insulin resistance is hepatic insulin resistance.

68. The method of embodiment 67, wherein the hepatic insulin resistance is caused by nonalcoholic fatty liver disease (NAFLD).

69. The compound or composition identified by the method of any one of embodiments 62-68.

70. A pharmaceutical composition comprising the compound or composition of embodiment 69 and at least one pharmaceutically acceptable diluent, excipient, or carrier.

71. A method of treating insulin resistance in a subject in need thereof, comprising administering the compound or composition of embodiment 69 or the pharmaceutical composition of embodiment 70 to the subject.

72. The method of embodiment 71, wherein the compound or composition or pharmaceutical composition is administered enterally, orally, parenterally, intravenously, intraperitoneally, intramuscularly, or subcutaneously.

73. The composition or composition identified by the method of any one of embodiments 62-68 for use in the treatment of insulin resistance in a subject in need thereof.

### BRIEF DESCRIPTION OF THE DRAWINGS

In addition to the features described herein, additional features and variations will be readily apparent from the following descriptions of the drawings and exemplary embodiments. It is to be understood that these drawings depict embodiments and are not intended to be limiting in scope.

FIG. 1A depicts an embodiment of a schematic representation of human liver organoid (HLO) formation.

FIG. 1B depicts an embodiment of bright field images of HLOs on day 20 of total culture.

FIG. 1C depicts an embodiment of immunofluorescent images of HLOs on day 20 of total culture stained with albumin (ALB), hepatocyte nuclear factor 4 alpha (HNF4), E-cadherin, and DAPI.

FIG. 2A depicts an embodiment of single cell RNA sequencing (scRNA-seq) profiling of hepatic cells in HLOs. Relative single cell expression levels for ALB, apolipoprotein E (APOE), collagen type 1 alpha 1 (COL1A1), and platelet derived growth factor receptor alpha (PDGFRA), which are characteristic hepatocyte markers, are shown.

FIG. 2B depicts an embodiment of relative single cell expression levels for ALB, retinol binding protein 4 (RBP4), tryptophan 2,3-dioxygenase (TDO2), COL1A1, actin alpha 2 (ACTA2), bone morphogenetic protein 4 (BMP4), keratin 19 (KRT19), wingless-type 6 (WNT6), and chromogranin A (CHGA), which are characteristic markers for hepatic stellate cells, biliary cells, and cholangiocytes, are shown.

FIG. 3A depicts an embodiment of a schematic representation of hepatic insulin response.

FIG. 3B depicts an embodiment of expression of receptor/signaling molecules required for insulin reaction in HLOs. Relative single cell expression levels for insulin receptor substrate 1 (IRS1), insulin receptor substrate 2 IRS2), and insulin receptor (INSR) are shown.

FIG. 3C depicts an embodiment of candidate reporter genes for assaying insulin responsiveness.

FIG. 4A depicts an embodiment of AKT phosphorylation in response to insulin treatment in HLOs.

FIG. 4B depicts an embodiment of suppression of gluconeogenesis regulatory gene expression in response to insulin treatment in HLOs. Relative expression levels of Forkhead box O1 (FOXO1), CAMP responsive element binding protein 1 (CREB1), TFE1, and phosphoenolpyruvate carboxykinase 1 (PCK1) are shown.

FIG. 4C depicts an embodiment induction of lipogenesis regulatory gene expression in response to insulin treatment in HLOs. Relative expression levels of sterol regulatory element-binding protein 1c (SREBP1C), fatty acid synthase (FASN), ATP-citrate lyase (ACLY), and acetyl-CoA carboxylase (ACC) are shown.

FIG. 5A depicts an embodiment of the establishment of PCK1 reporter iPSCs using CRISPR/Cas9 to visualize and quantify insulin response.

FIG. 5B depicts an embodiment of confirming reporter construct integration into iPSC clones.

FIG. 5C depicts an embodiment depicting that no change in cell morphology or proliferation is observed after PCK1 reporter construct integration.

FIG. 6A depicts an embodiment of immunofluorescent images showing upregulation of PCK1-dependent mScarlet fluorescence (corresponding to gluconeogenesis) following cAMP treatment, and downregulation of PCK1-dependent fluorescence following insulin treatment in HLOs.

FIG. 6B depicts an embodiment of upregulation of PCK1-dependent luciferase luminescence (corresponding to gluconeogenesis) following cAMP treatment and downregulation of PCK1-dependent luciferase luminescence following insulin treatment in HLOs.

FIG. 6C depicts an embodiment of detection of PCK1-luciferase luminescence in HLOs by in vitro imaging. The HLOs were treated with ± cAMP and ± insulin to modulate gluconeogenesis and insulin stimulation. HLOs that were not gene edited with the PCK1 reporter did not show any luminescence.

FIG. 6D depicts an embodiment of a quantification of PCK1-luciferase luminescence in HLOs detected with in vitro imaging of FIG. 6C.

FIG. 6E depicts an embodiment of testing the effect of different insulin concentrations (no insulin control, 10 nM, 100 nM, or 1000 nM) on PCK1-luciferase luminescence in previously insulin-starved HLOs. Luciferase activity of the PCK1 reporter was decreased in response to insulin stimulation.

FIG. 6F depicts an embodiment of testing the effect of different insulin treatment times (no insulin control, 1 hour, 2 hours, or 3 hours) on PCK1-luciferase luminescence in previously insulin-starved HLOs. Luciferase activity of the PCK1 reporter was reduced after 1 hour of treatment.

FIG. 6G depicts an embodiment of confirming the effects of cAMP treatment in previously insulin-starved HLOs. HLOs were treated with either 24 hours of cAMP, or 24 hours of cAMP followed by 3 hours of insulin before imaging. Luciferase activity of the PCK1 reporter was increased by cAMP treatment and inhibited by insulin stimulation.

FIG. 7A depicts an embodiment of a schematic representation of fatty liver HLO induction using oleic acid treatment.

FIG. 7B depicts an embodiment of immunofluorescent images depicting fat droplets and persistent PCK1 activation in fatty liver induced HLO generated using oleic acid.

FIG. 7C depicts an embodiment of quantification of triglycerides with NMR in control HLOs or steatohepatitis HLOs (sHLO) treated with 300 µM oleic acid.

FIG. 7D depicts an embodiment of an analysis of gene expression related to lipid droplet formation. Upregulation of DGAT1 and DGAT2, which catalyzes the formation of triglycerides from diacylgycerol and acyl-CoA, was detected in sHLO.

FIG. 7E depicts an embodiment of analysis of pro-inflammatory cytokine gene expression and secretion in control HLOs or sHLO. Gene expression of TNFa, TGFb, IL6, and IL8, and secretion of IL1b were tested. These pro-inflammatory cytokines were uniformly upregulated in sHLO.

FIG. 7F depicts an embodiment of PCK1-luciferase activity, which was upregulated in sHLO, as detected by in vitro imaging.

FIG. 7G depicts an embodiment of quantification of PCK1 expression as measured by luciferase luminescence (relative and absolute) and quantitative RT-PCR in fatty liver sHLO vs. control HLOs. sHLO exhibited increased expression and activity of PCK1.

FIG. 7H depicts an embodiment of quantification of glucose production in sHLO vs. control HLOs. The upregulation of PCK1 activity in sHLO was accompanied by enhanced glucose production.

FIG. 7I depicts an embodiment of an analysis of the insulin signal pathway in sHLO and control HLOs. Phosphorylation of AKT was inhibited in sHLO.

FIG. 7J depicts an embodiment of an analysis of insulin responsiveness in sHLO. Gene expression of downstream target genes of the insulin pathway was quantified. Insulin responsiveness of PCK1, CREB1, and FOXO1 was not suppressed in sHLO.

FIG. 7K depicts an embodiment of insulin response analysis of PCK1 in sHLO. PCK1 in sHLO was not responsive to insulin.

FIG. 7L depicts an embodiment of an analysis of insulin response on gluconeogenesis in sHLO. Glucose production in sHLO was not affected by insulin stimulation.

FIG. 8A depicts an embodiment of representative immunofluorescent images and quantification of reduced fat accumulation in fatty liver HLOs treated with obeticholic acid (OCA) in comparison to pioglitazone (PIO) and metformin (MET).

FIG. 8B depicts an embodiment of quantification of inflammation-related proteins in fatty liver HLOs treated with OCA, PIO and MET. Relative gene expression for tumor necrosis factor alpha (TNFα), nuclear factor kappa B subunit 1 (NFKB1), and nuclear factor kappa B subunit 2 (NFKB2) are shown.

FIG. 8C depicts an embodiment of restoration of insulin responsiveness in fatty liver HLOs following OCA treatment.

FIG. 8D depicts an embodiment of restoration of insulin responsiveness for representative genes involved in gluconeogenesis and lipogenesis in fatty liver HLOs following treatment with OCA. Relative gene expression for FOXO1, CREB, SREBP1C, and FASN are shown.

### DETAILED DESCRIPTION

In normal cells, stimulation with insulin generally reduces the rate of gluconeogenesis and increases the rate of lipogenesis. This occurs through the activation of a signaling pathway by the binding of insulin to the cell surface insulin receptor, and phosphorylation of AKT. Conversely, reduced levels of insulin and/or presence of either cAMP or glucagon results in the upregulation of gluconeogenesis. However, cells can develop insulin resistance and experience constitutive dysregulation of gluconeogenesis that is less perturbed by insulin. This phenomenon of insulin resistance is a significant medical burden that affects millions of individuals, most notably as prediabetes or type 2 diabetes, but also associated with other metabolic disorders.

The present disclosure relates generally to insulin resistance reporters that can be expressed in cells to provide an efficient and robust method to quantify an insulin response and associated gluconeogenesis and/or lipogenesis in the cells. This provides a rapid process of, for example, assessing the relative sensitivity of the cells to insulin or screening for compounds that affect insulin sensitivity or resistance exhibited by the cells. The cells may be derived from a patient, opening up the opportunity for personalized medicine.

Disclosed herein in some embodiments are insulin responsive cells, tissues, or organoids that comprise any one of the insulin resistance reporters disclosed herein. While all biological cells use insulin, notable cell types are those that relatively large energy consumers or serve to store and/or produce glucose, such as pancreatic cells, brain cells, adipose cells, muscle cells, or liver cells. The development of methods of producing organoids, which are three-dimensional cellular structures that closely resemble the morphology of living organs, also permit the study of tissue that more holistically represent natural biological functions. It is envisioned that the insulin resistance reporters can be engineered to any of these cells, tissues, or organoids for investigating insulin function in these cell types.

As the major location of gluconeogenesis in mammals is the liver, diseases affecting the liver may result in additional complications involving the insulin signaling pathway, gluconeogenesis, and lipogenesis. Comorbidity between fatty liver (e.g. NAFLD or NASH) and diabetes has been well recognized. Accordingly, there is a great need for an increased understanding of hepatic function in relation to these and other related diseases, as well as models for diagnosing and treating these diseases.

Accordingly, in some embodiments, the organoids are liver organoids, such as human liver organoids (HLOs). HLOs can be generated from pluripotent stem cells (PSCs), and the resultant liver organoid has proven to be superior to conventional two dimensional or three dimensional cultures of hepatocytes or “organoids” derived from adult stem cells, as PSC-derived organoids comprise liver tissue morphology and additional cell types other than hepatocytes, including mesenchymal cells, stellate cells, biliary cells, and cholangiocytes. Therefore, these PSC-derived HLOs are excellent models for investigating diseases associated with metabolism, insulin regulation, and inflammation. In some embodiments, these HLOs can be genetically engineered to express an insulin resistance reporter (such as any one of the insulin resistance reporters disclosed herein) and used as a model to recapitulate native human hepatic insulin response in vitro. In some embodiments, these HLOs can be further manipulated to exhibit a fatty liver phenotype. By comparing normal and fatty liver organoid function, assays to evaluate human hepatic insulin resistance are additionally described.

### Terms

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, technical and scientific terms used herein have the same meaning as commonly understood when read in light of the instant disclosure by one of ordinary skill in the art to which the present disclosure belongs. For purposes of the present disclosure, the following terms are explained below.

The disclosure herein uses affirmative language to describe the numerous embodiments. The disclosure also includes embodiments in which subject matter is excluded, in full or in part, such as substances or materials, method steps and conditions, protocols, or procedures.

The articles “a” and “an” are used herein to refer to one or to more than one (for example, at least one) of the grammatical object of the article. By way of example, “an element” means one element or more than one element.

By “about” is meant a quantity, level, value, number, frequency, percentage, dimension, size, amount, weight or length that varies by as much as 10% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “individual”, “subject”, or “patient” as used herein have their plain and ordinary meaning as understood in light of the specification, and mean a human or a non-human mammal, e.g., a dog, a cat, a mouse, a rat, a cow, a sheep, a pig, a goat, a non-human primate, or a bird, e.g., a chicken, as well as any other vertebrate or invertebrate. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, or the like.

The terms “effective amount” or “effective dose” as used herein have their plain and ordinary meaning as understood in light of the specification, and refer to that amount of a recited composition or compound that results in an observable effect. Actual dosage levels of active ingredients in an active composition of the presently disclosed subject matter can be varied so as to administer an amount of the active composition or compound that is effective to achieve the desired response for a particular subject and/or application. The selected dosage level will depend upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

The terms “function” and “functional” as used herein have their plain and ordinary meaning as understood in light of the specification, and refer to a biological, enzymatic, or therapeutic function.

The term “inhibit” as used herein has its plain and ordinary meaning as understood in light of the specification, and may refer to the reduction or prevention of a biological activity. The reduction can be by a percentage that is, is about, is at least, is at least about, is not more than, or is not more than about, 10%, 20%, 30%, 40%, 50%, 60%, 70%, 80%, 90%, or 100%, or an amount that is within a range defined by any two of the aforementioned values. As used herein, the term “delay” has its plain and ordinary meaning as understood in light of the specification, and refers to a slowing, postponement, or deferment of a biological event, to a time which is later than would otherwise be expected. The delay can be a delay of a percentage that is, is about, is at least, is at least about, is not more than, or is not more than about, 0%, 10%, 20%, 30%, 40%, 50%, 60%, 70%, 80%, 90%, 100%, or an amount within a range defined by any two of the aforementioned values. The terms inhibit and delay may not necessarily indicate a 100% inhibition or delay. A partial inhibition or delay may be realized.

As used herein, the term “isolated” has its plain and ordinary meaning as understood in light of the specification, and refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to, about, at least, at least about, not more than, or not more than about, 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are, are about, are at least, are at least about, are not more than, or are not more than about 80%, about 85%, about 90%, about 91%, about 92%, about 93%, about 94%, about 95%, about 96%, about 97%, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism or tissue.

As used herein, “in vivo” is given its plain and ordinary meaning as understood in light of the specification and refers to the performance of a method inside living organisms, usually animals, mammals, including humans, and plants, as opposed to a tissue extract or dead organism.

As used herein, “ex vivo” is given its plain and ordinary meaning as understood in light of the specification and refers to the performance of a method outside a living organism with little alteration of natural conditions.

As used herein, “in vitro” is given its plain and ordinary meaning as understood in light of the specification and refers to the performance of a method outside of biological conditions, e.g., in a petri dish or test tube.

The terms “nucleic acid” or “nucleic acid molecule” as used herein have their plain and ordinary meaning as understood in light of the specification, and refer to polynucleotides, such as deoxyribonucleic acid (DNA) or ribonucleic acid (RNA), oligonucleotides, those that appear in a cell naturally, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate. The term “nucleic acid molecule” also includes so-called “peptide nucleic acids,” which comprise naturally-occurring or modified nucleic acid bases attached to a polyamide backbone. Nucleic acids can be either single stranded or double stranded. “Oligonucleotide” can be used interchangeable with nucleic acid and can refer to either double stranded or single stranded DNA or RNA. A nucleic acid or nucleic acids can be contained in a nucleic acid vector or nucleic acid construct (e.g. plasmid, virus, retrovirus, lentivirus, bacteriophage, cosmid, fosmid, phagemid, bacterial artificial chromosome (BAC), yeast artificial chromosome (YAC), or human artificial chromosome (HAC)) that can be used for amplification and/or expression of the nucleic acid or nucleic acids in various biological systems. Typically, the vector or construct will also contain elements including but not limited to promoters, enhancers, terminators, inducers, ribosome binding sites, translation initiation sites, start codons, stop codons, polyadenylation signals, origins of replication, cloning sites, multiple cloning sites, restriction enzyme sites, epitopes, reporter genes, selection markers, antibiotic selection markers, targeting sequences, peptide purification tags, or accessory genes, or any combination thereof.

A nucleic acid or nucleic acid molecule can comprise one or more sequences encoding different peptides, polypeptides, or proteins. These one or more sequences can be joined in the same nucleic acid or nucleic acid molecule adj acently, or with extra nucleic acids in between, e.g. linkers, repeats or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a nucleic acid as used herein has its plain and ordinary meaning as understood in light of the specification and refers to a sequence being after the 3′-end of a previous sequence, on the strand containing the encoding sequence (sense strand) if the nucleic acid is double stranded. The term “upstream” on a nucleic acid as used herein has its plain and ordinary meaning as understood in light of the specification and refers to a sequence being before the 5′-end of a subsequent sequence, on the strand containing the encoding sequence (sense strand) if the nucleic acid is double stranded. The term “grouped” on a nucleic acid as used herein has its plain and ordinary meaning as understood in light of the specification and refers to two or more sequences that occur in proximity either directly or with extra nucleic acids in between, e.g. linkers, repeats, or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths, but generally not with a sequence in between that encodes for a functioning or catalytic polypeptide, protein, or protein domain.

The nucleic acids described herein comprise nucleobases. Primary, canonical, natural, or unmodified bases are adenine, cytosine, guanine, thymine, and uracil. Other nucleobases include but are not limited to purines, pyrimidines, modified nucleobases, 5-methylcytosine, pseudouridine, dihydrouridine, inosine, 7-methylguanosine, hypoxanthine, xanthine, 5,6-dihydrouracil, 5-hydroxymethylcytosine, 5-bromouracil, isoguanine, isocytosine, aminoallyl bases, dye-labeled bases, fluorescent bases, or biotin-labeled bases.

The terms “peptide”, “polypeptide”, and “protein” as used herein have their plain and ordinary meaning as understood in light of the specification and refer to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a polypeptide as used herein has its plain and ordinary meaning as understood in light of the specification and refers to a sequence being after the C-terminus of a previous sequence. The term “upstream” on a polypeptide as used herein has its plain and ordinary meaning as understood in light of the specification and refers to a sequence being before the N-terminus of a subsequent sequence.

The term “purity” of any given substance, compound, or material as used herein has its plain and ordinary meaning as understood in light of the specification and refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to nucleic acids, DNA, RNA, nucleotides, proteins, polypeptides, peptides, amino acids, lipids, cell membrane, cell debris, small molecules, degradation products, solvent, carrier, vehicle, or contaminants, or any combination thereof. In some embodiments, the substance, compound, or material is substantially free of host cell proteins, host cell nucleic acids, plasmid DNA, contaminating viruses, proteasomes, host cell culture components, process related components, mycoplasma, pyrogens, bacterial endotoxins, and adventitious agents. Purity can be measured using technologies including but not limited to electrophoresis, SDS-PAGE, capillary electrophoresis, PCR, rtPCR, qPCR, chromatography, liquid chromatography, gas chromatography, thin layer chromatography, enzyme-linked immunosorbent assay (ELISA), spectroscopy, UV-visible spectrometry, infrared spectrometry, mass spectrometry, nuclear magnetic resonance, gravimetry, or titration, or any combination thereof.

The term “yield” of any given substance, compound, or material as used herein has its plain and ordinary meaning as understood in light of the specification and refers to the actual overall amount of the substance, compound, or material relative to the expected overall amount. For example, the yield of the substance, compound, or material is, is about, is at least, is at least about, is not more than, or is not more than about, 80, 85, 90, 91, 92, 93, 94, 95, 96, 97, 98, 99, or 100% of the expected overall amount, including all decimals in between. Yield may be affected by the efficiency of a reaction or process, unwanted side reactions, degradation, quality of the input substances, compounds, or materials, or loss of the desired substance, compound, or material during any step of the production.

The term “insulin” as used herein has its plain and ordinary meaning as understood in light of the specification and refers to the major metabolic hormone that regulates glucose uptake in cells of the body. Insulin typically interacts with the transmembrane insulin receptor (INSR) protein found on cell surfaces. Binding of insulin to the receptor induces a signal transduction pathway that imparts effects including but not limited to glycogenesis in liver cells, uptake of blood glucose in cells such as muscle and adipose cells, downregulation of gluconeogenesis, and upregulation of lipogenesis. In some embodiments, the methods involving contacting or treating with insulin can be also done with an insulin derivative or mimetic thereof, including but not limited to insulin, insulin aspart, insulin glulisine, insulin lispro, insulin isophane, insulin degludec, insulin detemir, insulin zinc, or insulin glargine. Additionally, other compounds or compositions having effects on glucose metabolism and/or regulation, insulin sensitivity or used for the treatment of diabetes, including but not limited to, vanadium, biguanides, metformin, phenformin, buformin, thiazolidinediones, rosiglitazone, pioglitazone, troglitazone, tolimidone, sulfonylureas, tolbutamide, acetohexamide, tolazamide, chlorpropamide, glipizide, glibenclamide, glimepiride, gliclazide, glyclopyramide, gliquidone, meglitinides, repaglinide, nateglinide, alpha-glucosidase inhibitors, miglitol, acarbose, voglibose, incretins, glucagon-like peptide 1, glucagon-like peptide agonists, exenatide, liraglutide, taspoglutide, lixisenatide, semaglutide, dulaglutide, gastric inhibitory peptide, dipeptidyl peptidase-4 inhibitors, vildagliptin, sitagliptin, saxagliptin, linagliptin, alogliptin, septagliptin, teneligpliptin, gemigliptin, pramlintide, dapagliflozin, canagliflozin, empagliflozin, or remogliflozin can also be used as a substitute or in combination with the insulin or derivative or mimetics thereof listed herein.

The term “insulin responsive” as used herein has its plain and ordinary meaning as understood in light of the specification and refers to a cell, tissue or organoid that produces, interacts, or reacts to insulin. Insulin responsive cells, tissue, or organoids include but are not limited to pancreatic cells, brain cells, adipose cells, muscle cells, or liver cells, or any tissue or organoid that comprise one or more of the cells thereof. Dysfunctional insulin response (e.g. insulin resistance or hypersensitivity) of these cells or tissues can cause or is attributed with numerous diseases or disorders in patients, including but not limited to diabetes, hyperinsulinemia, weight gain, hypertension, hyperglycemia, dyslipidemia, or inflammatory diseases such as nonalcoholic fatty liver disease (NALFD). In some embodiments, any one of the methods described herein using insulin responsive cells, tissues, or organoids apply to the cells, or tissues or organoids thereof, listed herein. Furthermore, in other embodiments, any one of the stem cells or stem cell compositions described herein can be differentiated into any one of the cells, or tissues or organoids thereof, listed herein.

The term “insulin resistance” as used herein has its plain and ordinary meaning as understood in light of the specification and refers to the phenomenon where cells become less sensitive to insulin. This applies to individuals as well, where the insulin can be produced endogenously by the pancreas of the individual, or administered exogenously for treatments. Excessive blood sugar levels and/or increased blood levels of insulin (which may also arise due to the pancreas responding to high blood sugar levels) may lead to cells becoming less responsive to normal levels of insulin release. This can notably lead to prediabetes and type 2 diabetes, but insulin resistance is associated with other diseases and disorders, such as metabolic syndromes, fatty liver disease, steatohepatitis, obesity, cardiovascular disease, polycystic ovary syndrome, hyperglycemia, hyperinsulinemia, or dyslipidemia. Furthermore, while insulin resistance is not generally the main cause of type 1 diabetes, type 1 diabetics can also develop insulin resistance.

The term “insulin resistance reporter” as used herein has its plain and ordinary meaning as understood in light of the specification and refers to a biologically relevant construct that reacts to changes in insulin levels, activity of an insulin-dependent pathway, or any target or pathway that is affected by activity of insulin, and presents a detectable effect in accordance with said reaction. It will be understood by the skilled person that this term may apply to a nucleic acid construct which might or might not directly act as the reporter, but translate into protein(s) that perform the function of the reporter, or apply to the protein(s) directly. Exemplary reporters that are commonly used in the art and used herein include reporters that emit light, which can be easily captured with conventional microscopes and other devices. However, it is envisioned that other reporters known in the art, such as chemical reporters, which induce a chemical reaction in response to the stimulus (e.g. blue-white screening) or selectable marker reporters (e.g. antibiotic resistance) can be used as well, either in combination with or substituting for the other reporters. As disclosed herein, one approach to generate an insulin resistance reporter is to operatively link expression of one or more reporters to the expression of a protein involved in an insulin related pathway. In this instance, modulation of expression of the protein involved in the insulin related pathway (e.g. upregulation or downregulation of expression) will also lead to the same modulation of the reporters.

It will be understood that the term “insulin resistance reporter” as used herein is not limited to reporters that detect only the phenomenon of insulin resistance. Instead, “insulin resistance reporter” is intended to encompass any embodiments of reporter constructs that can present a detectable effect in response to any modulation of insulin activity, expression, abundance, or function, including insulin sensitivity. Some non-limiting examples of applications of the insulin resistance reporters disclosed herein include modeling of insulin activity under physiologic, normal conditions, modeling of insulin resistance, modeling of insulin hypersensitivity, and post-transplant monitoring of a subject. The term “insulin response reporter” is used interchangeably with “insulin resistance reporter” herein.

The term “insulin-dependent gene” as used herein has its plain and ordinary meaning as understood in light of the specification and refers to any gene (and the respectively expressed protein) that is involved in an insulin-related pathway. This gene may be involved in the insulin signaling cascade, where binding of insulin to an insulin receptor triggers activation or inhibition of activity of certain metabolic enzymes, such as PCK1, which may also entail post-translational modifications of proteins (e.g. phosphorylation of AKT), or changes in expression of regulatory genes. Genes and proteins that regulate or carry out gluconeogenesis and lipogenesis are included in the category of insulin-dependent gene, as these two metabolic processes are both affected by insulin signaling in the cell. It will be understood that operatively linking any one insulin-dependent gene to an insulin resistance reporter would permit the measurement of the insulin-dependent gene and detection of any change in abundance or function of the insulin-dependent gene in response to insulin stimulation, or lack thereof. It is envisioned that a skilled person will understand the intended outcome of using a particular insulin-dependent gene. For example, a protein involved in gluconeogenesis would be expected to exhibit a reduction in expression in response to insulin, whereas a protein involved in lipogenesis would be expected to increase in expression in response to insulin.

The term “bicistronic element” as used herein has its plain and ordinary meaning as understood in light of the specification and refers to a genetic sequence that results in the expression of two separate proteins, based on the coding sequences flanking said bicistronic element, rather than the production of a fusion of the two proteins. Two major bicistronic elements are generally known: self-cleaving peptides and internal ribosome entry sites (IRES). Self-cleaving peptides take advantage of a certain sequence of amino acids that results in ribosomal skipping of the creation of a peptide bond between two amino acids of the sequence. This results in two proteins that are not bound together after translation. Well known self-cleaving peptides include the T2A, P2A, E2A, and F2A sequences. IRES involve an RNA sequence that forms a secondary structure sufficient to recruit a ribosome without the normal 5′ cap. Both of these bicistronic element types are useful where expression of two or more separate proteins are desirable, and where the relative levels of the two or more separate proteins are generally kept equivalent, because both arise from the same mRNA. As disclosed herein, bicistronic elements can be placed between an insulin-dependent gene and an insulin resistance reporter to induce equivalent expression of both components such that measurement of levels of the insulin resistance reporter will allow inference towards the levels of the insulin-dependent gene. Furthermore, bicistronic elements can be placed between two or more reporter genes so that multiple approaches of detection are possible (e.g. fluorescence and luminescence).

As used herein, “pharmaceutically acceptable” has its plain and ordinary meaning as understood in light of the specification and refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and are intended to include any and all solvents, dispersion media, coatings, antibacterial and antifungal agents, isotonic and absorption delaying agents, and the like, compatible with administration to humans, cats, dogs, or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals, such as cats and dogs. The term diluent, excipient, and/or “carrier” can refer to a diluent, adjuvant, excipient, or vehicle with which the pharmaceutical composition is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. A non-limiting example of a physiologically acceptable carrier is an aqueous pH buffered solution. The physiologically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as mannitol or sorbitol, salt-forming counterions such as sodium, and nonionic surfactants such as TWEEN®, polyethylene glycol (PEG), and PLURONICS®. The composition, if desired, can also contain minor amounts of wetting, bulking, emulsifying agents, or pH buffering agents. These compositions can take the form of solutions, suspensions, emulsion, sustained release formulations and the like. The formulation should suit the mode of administration.

Cryoprotectants are cell composition additives to improve efficiency and yield of low temperature cryopreservation by preventing formation of large ice crystals. Cryoprotectants include but are not limited to DMSO, ethylene glycol, glycerol, propylene glycol, trehalose, formamide, methyl-formamide, dimethyl-formamide, glycerol 3-phosphate, proline, sorbitol, diethyl glycol, sucrose, triethylene glycol, polyvinyl alcohol, polyethylene glycol, or hydroxyethyl starch. Cryoprotectants can be used as part of a cryopreservation medium, which include other components such as nutrients (e.g. albumin, serum, bovine serum, fetal calf serum [FCS]) to enhance post-thawing survivability of the cells. In these cryopreservation media, at least one cryoprotectant may be found at a concentration that is, is about, is at least, is at least about, is not more than, or is not more than about, 0.01%, 0.05%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90%, or any percentage within a range defined by any two of the aforementioned numbers.

Additional excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, fructose, mannose, lactose, galactose, sucrose, sorbitol, cellulose, serum, amino acids, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. Some excipients may be in residual amounts or contaminants from the process of manufacturing, including but not limited to serum, albumin, ovalbumin, antibiotics, inactivating agents, formaldehyde, glutaraldehyde, β-propiolactone, gelatin, cell debris, nucleic acids, peptides, amino acids, or growth medium components or any combination thereof. The amount of the excipient may be found in composition at a percentage that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “pharmaceutically acceptable salts” has its plain and ordinary meaning as understood in light of the specification and includes relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; trihydroxymethyl aminoethane.

Proper formulation is dependent upon the route of administration chosen. Techniques for formulation and administration of the compounds described herein are known to those skilled in the art. Multiple techniques of administering a compound exist in the art including, but not limited to, enteral, oral, rectal, topical, sublingual, buccal, intraaural, epidural, epicutaneous, aerosol, parenteral delivery, including intramuscular, subcutaneous, intra-arterial, intravenous, intraportal, intra-articular, intradermal, peritoneal, intramedullary injections, intrathecal, direct intraventricular, intraperitoneal, intranasal or intraocular injections. Pharmaceutical compositions will generally be tailored to the specific intended route of administration.

As used herein, a “carrier” has its plain and ordinary meaning as understood in light of the specification and refers to a compound, particle, solid, semi-solid, liquid, or diluent that facilitates the passage, delivery and/or incorporation of a compound to cells, tissues and/or bodily organs.

As used herein, a “diluent” has its plain and ordinary meaning as understood in light of the specification and refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

The term “% w/w” or “% wt/wt” as used herein has its plain and ordinary meaning as understood in light of the specification and refers to a percentage expressed in terms of the weight of the ingredient or agent over the total weight of the composition multiplied by 100. The term “% v/v” or “% vol/vol” as used herein has its plain and ordinary meaning as understood in the light of the specification and refers to a percentage expressed in terms of the liquid volume of the compound, substance, ingredient, or agent over the total liquid volume of the composition multiplied by 100.

### Stem Cells

The term “totipotent stem cells” (also known as omnipotent stem cells) as used herein has its plain and ordinary meaning as understood in light of the specification and are stem cells that can differentiate into embryonic and extra-embryonic cell types. Such cells can construct a complete, viable organism. These cells are produced from the fusion of an egg and sperm cell. Cells produced by the first few divisions of the fertilized egg are also totipotent.

The term “embryonic stem cells (ESCs),” also commonly abbreviated as ES cells, as used herein has its plain and ordinary meaning as understood in light of the specification and refers to cells that are pluripotent and derived from the inner cell mass of the blastocyst, an early-stage embryo. For purpose of the present disclosure, the term “ESCs” is used broadly sometimes to encompass the embryonic germ cells as well.

The term “pluripotent stem cells (PSCs)” as used herein has its plain and ordinary meaning as understood in light of the specification and encompasses any cells that can differentiate into nearly all cell types of the body, i.e., cells derived from any of the three germ layers (germinal epithelium), including endoderm (interior stomach lining, gastrointestinal tract, the lungs), mesoderm (muscle, bone, blood, urogenital), and ectoderm (epidermal tissues and nervous system). PSCs can be the descendants of inner cell mass cells of the preimplantation blastocyst or obtained through induction of a non-pluripotent cell, such as an adult somatic cell, by forcing the expression of certain genes. Pluripotent stem cells can be derived from any suitable source. Examples of sources of pluripotent stem cells include mammalian sources, including human, rodent, porcine, and bovine.

The term “induced pluripotent stem cells (iPSCs),” also commonly abbreviated as iPS cells, as used herein has its plain and ordinary meaning as understood in light of the specification and refers to a type of pluripotent stem cells artificially derived from a normally non-pluripotent cell, such as an adult somatic cell, by inducing a “forced” expression of certain genes. hiPSC refers to human iPSCs. In some methods known in the art, iPSCs may be derived by transfection of certain stem cell-associated genes into non-pluripotent cells, such as adult fibroblasts. Transfection may be achieved through viral transduction using viruses such as retroviruses or lentiviruses. Transfected genes may include the master transcriptional regulators Oct-¾ (POU5F1) and Sox2, although other genes may enhance the efficiency of induction. After 3-4 weeks, small numbers of transfected cells begin to become morphologically and biochemically similar to pluripotent stem cells, and are typically isolated through morphological selection, doubling time, or through a reporter gene and antibiotic selection. As used herein, iPSCs include first generation iPSCs, second generation iPSCs in mice, and human induced pluripotent stem cells. In some methods, a retroviral system is used to transform human fibroblasts into pluripotent stem cells using four pivotal genes: Oct¾, Sox2, Klf4, and c-Myc. In other methods, a lentiviral system is used to transform somatic cells with OCT4, SOX2, NANOG, and LIN28. Genes whose expression are induced in iPSCs include but are not limited to Oct-¾ (POU5F1); certain members of the Sox gene family (e.g., Soxl, Sox2, Sox3, and Sox15); certain members of the Klf family (e.g., Klfl, Klf2, Klf4, and Klf5), certain members of the Myc family (e.g., C-myc, L-myc, and N-myc), Nanog, LIN28, Tert, Fbx15, ERas, ECAT15-1, ECAT15-2, Tcl1, β-Catenin, ECAT1, Esg1, Dnmt3L, ECAT8, Gdf3, Fth117, Sal14, Rex1, UTF1, Stella, Stat3, Grb2, Prdm14, Nr5a1, Nr5a2, or E-cadherin, or any combination thereof.

The term “precursor cell” as used herein has its plain and ordinary meaning as understood in light of the specification and encompasses any cells that can be used in methods described herein, through which one or more precursor cells acquire the ability to renew itself or differentiate into one or more specialized cell types. In some embodiments, a precursor cell is pluripotent or has the capacity to becoming pluripotent. In some embodiments, the precursor cells are subjected to the treatment of external factors (e.g., growth factors) to acquire pluripotency. In some embodiments, a precursor cell can be a totipotent (or omnipotent) stem cell; a pluripotent stem cell (induced or non-induced); a multipotent stem cell; an oligopotent stem cells and a unipotent stem cell. In some embodiments, a precursor cell can be from an embryo, an infant, a child, or an adult. In some embodiments, a precursor cell can be a somatic cell subject to treatment such that pluripotency is conferred via genetic manipulation or protein/peptide treatment. Precursor cells include embryonic stem cells (ESC), embryonic carcinoma cells (ECs), and epiblast stem cells (EpiSC).

In some embodiments, one step is to obtain stem cells that are pluripotent or can be induced to become pluripotent. In some embodiments, pluripotent stem cells are derived from embryonic stem cells, which are in turn derived from totipotent cells of the early mammalian embryo and are capable of unlimited, undifferentiated proliferation in vitro. Embryonic stem cells are pluripotent stem cells derived from the inner cell mass of the blastocyst, an early-stage embryo. Methods for deriving embryonic stem cells from blastocytes are well known in the art. Human embryonic stem cells H9 (H9-hESCs) are used in the exemplary embodiments described in the present application, but it would be understood by one of skill in the art that the methods and systems described herein are applicable to any stem cells.

Additional stem cells that can be used in embodiments in accordance with the present disclosure include but are not limited to those provided by or described in the database hosted by the National Stem Cell Bank (NSCB), Human Embryonic Stem Cell Research Center at the University of California, San Francisco (UCSF); WISC cell Bank at the Wi Cell Research Institute; the University of Wisconsin Stem Cell and Regenerative Medicine Center (UW-SCRMC); Novocell, Inc. (San Diego, Calif.); Cellartis AB (Goteborg, Sweden); ES Cell International Pte Ltd (Singapore); Technion at the Israel Institute of Technology (Haifa, Israel); and the Stem Cell Database hosted by Princeton University and the University of Pennsylvania. Exemplary embryonic stem cells that can be used in embodiments in accordance with the present disclosure include but are not limited to SA01 (SA001); SA02 (SA002); ES01 (HES-1); ES02 (HES-2); ES03 (HES-3); ES04 (HES-4); ES05 (HES-5); ES06 (HES-6); BG01 (BGN-01); BG02 (BGN-02); BG03 (BGN-03); TE03 (13); TE04 (14); TE06 (16); UCOl (HSF1); UC06 (HSF6); WA01 (HI); WA07 (H7); WA09 (H9); WA13 (H13); WA14 (H14). Exemplary human pluripotent cell lines include but are not limited to TkDA3-4, 1231A3, 317-D6, 317-A4, CDH1, 5-T-3, 3-34-1, NAFLD27, NAFLD77, NAFLD150, WD90, WD91, WD92, L20012, C213, 1383D6, FF, or 317-12 cells.

In developmental biology, cellular differentiation is the process by which a less specialized cell becomes a more specialized cell type. As used herein, the term “directed differentiation” describes a process through which a less specialized cell becomes a particular specialized target cell type. The particularity of the specialized target cell type can be determined by any applicable methods that can be used to define or alter the destiny of the initial cell. Exemplary methods include but are not limited to genetic manipulation, chemical treatment, protein treatment, and nucleic acid treatment.

In some embodiments, an adenovirus can be used to transport the requisite four genes, resulting in iPSCs substantially identical to embryonic stem cells. Since the adenovirus does not combine any of its own genes with the targeted host, the danger of creating tumors is eliminated. In some embodiments, non-viral based technologies are employed to generate iPSCs. In some embodiments, reprogramming can be accomplished via plasmid without any virus transfection system at all, although at very low efficiencies. In other embodiments, direct delivery of proteins is used to generate iPSCs, thus eliminating the need for viruses or genetic modification. In some embodiment, generation of mouse iPSCs is possible using a similar methodology: a repeated treatment of the cells with certain proteins channeled into the cells via poly-arginine anchors was sufficient to induce pluripotency. In some embodiments, the expression of pluripotency induction genes can also be increased by treating somatic cells with FGF2 under low oxygen conditions.

The term “feeder cell” as used herein has its plain and ordinary meaning as understood in light of the specification and refers to cells that support the growth of pluripotent stem cells, such as by secreting growth factors into the medium or displaying on the cell surface. Feeder cells are generally adherent cells and may be growth arrested. For example, feeder cells are growth-arrested by irradiation (e.g. gamma rays), mitomycin-C treatment, electric pulses, or mild chemical fixation (e.g. with formaldehyde or glutaraldehyde). However, feeder cells do not necessarily have to be growth arrested. Feeder cells may serve purposes such as secreting growth factors, displaying growth factors on the cell surface, detoxifying the culture medium, or synthesizing extracellular matrix proteins. In some embodiments, the feeder cells are allogeneic or xenogeneic to the supported target stem cell, which may have implications in downstream applications. In some embodiments, the feeder cells are mouse cells. In some embodiments, the feeder cells are human cells. In some embodiments, the feeder cells are mouse fibroblasts, mouse embryonic fibroblasts, mouse STO cells, mouse 3T3 cells, mouse SNL 76/7 cells, human fibroblasts, human foreskin fibroblasts, human dermal fibroblasts, human adipose mesenchymal cells, human bone marrow mesenchymal cells, human amniotic mesenchymal cells, human amniotic epithelial cells, human umbilical cord mesenchymal cells, human fetal muscle cells, human fetal fibroblasts, or human adult fallopian tube epithelial cells. In some embodiments, conditioned medium prepared from feeder cells is used in lieu of feeder cell co-culture or in combination with feeder cell co-culture. In some embodiments, feeder cells are not used during the proliferation of the target stem cells.

### Gene Editing

Any of the cells disclosed herein, such as stem cells, pluripotent stem cells, iPSCs, ESCs, definitive endoderm cells, foregut endoderm cells, anterior foregut cells (or anterior foregut spheroids), or organoids (including but not limited to liver organoids), can be genetically modified to express an insulin resistance reporter. In some embodiments, the iPSCs or ESCs are genetically modified before differentiation into definitive endoderm cells, anterior foregut spheroids, or organoids, or any combination thereof. In some embodiments, the iPSCs are first differentiated into definitive endoderm cells before genetic modification. In some embodiments, the definitive endoderm cells are genetically modified before differentiation into anterior foregut spheroids or organoids, or both. In some embodiments, the definitive endoderm cells are first differentiated into anterior foregut spheroids before genetic modification. In some embodiments, the anterior foregut spheroids are genetically modified before differentiation into organoids. In some embodiments, the anterior foregut spheroids are differentiation into organoids before genetic modification. In some embodiments, the organoids are genetically modified.

The cells disclosed herein can be modified with the insulin resistance reporter using methods generally known in the art. In some embodiments, the cells are genetically modified using a CRISPR nuclease, TALEN, zinc finger nuclease, meganuclease, or megaTAL. In some embodiments, the cells can be genetically modified using a non-homologous end joining or homology directed repair approach. In some embodiments, the cells are genetically modified using a CRISPR nuclease. In some embodiments, the cells are genetically modified using a homology approach. In some embodiments, the cells are genetically modified using a homology approach with a CRISPR nuclease. In some embodiments, the CRISPR nuclease is Cas9, Cpf1, Cas12a, Cas12b, Cas13a, Cas13b, Cas13c, Cas13d, or Cas14a. In some embodiments, the CRISPR nuclease is Cas9.

In some embodiments, the cells are genetically modified with an insulin resistance reporter. In some embodiments, the insulin resistance reporter is a gluconeogenesis reporter. In some embodiments, the insulin resistance reporter is a lipogenesis reporter. In some embodiments, the insulin resistance reporter comprises one or more nucleic acid sequences encoding for a reporter protein. In some embodiments, the reporter protein is a fluorescent protein. In some embodiments, the insulin resistance reporter comprises one or more nucleic acid sequences encoding for a fluorescent protein. In some embodiments, the fluorescent protein is mScarlet. In some embodiments, mScarlet is encoded by the nucleic acid sequence of SEQ ID NO: 8. In some embodiments, mScarlet comprises the peptide sequence of SEQ ID NO: 15. In some embodiments, the reporter protein is a luminescent protein. In some embodiments, the insulin resistance reporter comprises one or more nucleic acid sequences encoding for a luminescent protein. In some embodiments, the luminescent protein is luciferase. In some embodiments, luciferase is encoded by the nucleic acid sequence of SEQ ID NO: 10. In some embodiments, luciferase comprises the peptide sequence of SEQ ID NO: 17. In some embodiments, the insulin resistance reporter comprises one or more nucleic acid sequences encoding for a resistance marker. In some embodiments, the resistance marker is a neomycin resistance marker (neoR). In some embodiments, the neomycin resistance marker is encoded by the nucleic acid sequence of SEQ ID NO: 11. In some embodiments, the neomycin resistance marker comprises the peptide sequence of SEQ ID NO: 18. In some embodiments, the insulin resistance reporter comprise one or more bicistronic elements. In some embodiments, the bicistronic element is a self-cleaving peptide or an IRES. In some embodiments, the insulin resistance reporter comprises one or more nucleic acid sequences encoding for a self-cleaving peptide. In some embodiments, the one or more self-cleaving peptides are P2A, T2A, E2A, or F2A, or any combination thereof. In some embodiments, the P2A self-cleaving peptide comprises the nucleic acid sequence of SEQ ID NO: 7 and the peptide sequence of SEQ ID NO: 14. In some embodiments, the T2A self-cleaving peptide comprises the nucleic acid sequence of SEQ ID NO: 9 and the peptide sequence of SEQ ID NO: 16. In some embodiments, the insulin resistance reporter comprises one or more nucleic acid sequences having homology to an endogenous gene. In some embodiments, the endogenous gene is a gene involved in gluconeogenesis or lipogenesis. In some embodiments, the gene is PCK1. In some embodiments, the gene is selected from the group consisting of PCK1, G6PC, G6PC2, G6PC3, FOXO1, CREB1, GSK3A, GSK3B, MTOR, SREBP1C, ACC, ACLY, FASN, and GCK. In some embodiments, the insulin resistance reporter comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 4. In some embodiments, the insulin resistance reporter is inserted to the endogenous gene in the cell such that the expression the endogenous gene results in expression of the fluorescent protein, the luminescent protein, the resistance marker, or any combination thereof.

In some embodiments, the cells are genetically modified with an insulin resistance reporter. In some embodiments, the insulin resistance reporter is operatively linked to an insulin-dependent gene of the cell. In some embodiments, the insulin-dependent gene is a gluconeogenesis gene or a lipogenesis gene. In some embodiments, the insulin-dependent gene is any gene (or resultant protein) that is modulated in some way in response to insulin signaling. In some embodiments, the insulin-dependent gene is selected from the group consisting of PCK1, G6PC, G6PC2, G6PC3, GSK3A, GSK3B, MTOR, GCK, FOXO1, CREB1, TFE1, TFE3, SREBP1C, FASN, ACLY, and ACC. In some embodiments, the insulin-dependent gene is PCK1. In some embodiments, the expression of the insulin-dependent gene results in expression of the insulin resistance reporter. In some embodiments, the insulin-dependent gene and the insulin resistance reporter are separated by a bicistronic element. In some embodiments, the bicistronic element is a self-cleaving peptide or an IRES. In some embodiments, the self-cleaving peptide is selected from P2A, T2A, E2A, or F2A, or any combination thereof. In some embodiments, the P2A self-cleaving peptide comprises the nucleic acid sequence of SEQ ID NO: 7 and the peptide sequence of SEQ ID NO: 14. In some embodiments, the T2A self-cleaving peptide comprises the nucleic acid sequence of SEQ ID NO: 9 and the peptide sequence of SEQ ID NO: 16. In some embodiments, the insulin resistance reporter has been integrated at the locus of the insulin-dependent gene. In some embodiments, the insulin resistance reporter has been integrated at the locus of the insulin-dependent gene using a CRISPR nuclease. In some embodiments, the CRISPR nuclease is Cas9, Cpf1, Cas12a, Cas12b, Cas13a, Cas13b, Cas13c, Cas13d, or Cas14a. In some embodiments, the CRISPR nuclease is Cas9. In some embodiments, the insulin resistance reporter is integrated at the locus of the insulin-dependent gene using homology directed repair. Exemplary regions of homology for PCK1 for integration of the insulin resistance reporter by homology directed repair are provided as SEQ ID NO: 6 and 13. In some embodiments, the insulin resistance reporter comprises two or more reporter genes, and the two or more reporter genes are separated by one or more bicistronic elements.

In some embodiments, the iPSCs, definitive endoderm cells, anterior foregut spheroids, or organoids are genetically modified or edited according to methods known in the art. For example, gene editing using CRISPR nucleases such as Cas9 are explored in PCT Publications WO 2013/176772, WO 2014/093595, WO 2014/093622, WO 2014/093655, WO 2014/093712, WO 2014/093661, WO 2014/204728, WO 2014/204729, WO 2015/071474, WO 2016/115326, WO 2016/141224, WO 2017/023803, and WO 2017/070633, each of which is hereby expressly incorporated by reference in its entirety.

### Insulin Resistance Reporters

Disclosed herein are insulin resistance reporters. These reporters are embodied as nucleic acid constructs and resultant expressed proteins that are used to visualize, measure, or quantify a system associated with gluconeogenesis, lipogenesis, or other pathway associated with insulin activity in a biological cell. Generally, the insulin resistance reporters disclosed herein function by expression of one or more reporter proteins that occurs with expression of a protein that is involved in an insulin pathway, where changes in expression of the insulin-related protein will also apply to the expression of the one or more reporter proteins.

Provided herein are insulin resistance reporters comprising one or more reporter genes flanked by a 5′ homology region and a 3′ homology region associated with an insulin-dependent gene. The 5′ homology region and 3′ homology region are used for homology directed gene editing of the one or more reporter genes at the locus of the insulin-dependent gene. In some embodiments, the insulin-dependent gene is a gluconeogenesis gene or a lipogenesis gene. In some embodiments, the insulin-dependent gene is selected from the group consisting of PCK1, G6PC, G6PC2, G6PC3, GSK3A, GSK3B, MTOR, GCK, FOXO1, CREB1, TFE1, TFE3, SREBP1C, FASN, ACLY, and ACC. In some embodiments, the insulin-dependent gene is PCK1. In some embodiments, at least one of the one or more reporter genes and the 5′ homology region associated with an insulin-dependent gene are separated by a bicistronic element. In some embodiments, the insulin resistance reporter is intended to be inserted 3′ of the insulin-dependent gene. In some embodiments, at least one of the one or more reporter genes and the 3′ homology region associated with an insulin-dependent gene are separated by a bicistronic element. In some embodiments, the insulin resistance reporter is intended to be inserted 5′ of the insulin-dependent gene. In some embodiments, the bicistronic element is a self-cleaving peptide or an IRES. In some embodiments, the self-cleaving peptide is a P2A, T2A, E2A, or F2A self-cleaving peptide. In some embodiments, the one or more reporter genes comprise a gene encoding for a fluorescent protein or a gene encoding for a luminescent protein, or both. In some embodiments, the fluorescent protein comprises mScarlet or the luminescent protein comprises luciferase. However, any other fluorescent protein and any other luminescent protein generally known in the art can be used. In some embodiments, mScarlet is encoded by the nucleic acid sequence of SEQ ID NO: 8. In some embodiments, mScarlet comprises the peptide sequence of SEQ ID NO: 15. In some embodiments, luciferase is encoded by the nucleic acid sequence of SEQ ID NO: 10. In some embodiments, luciferase comprises the peptide sequence of SEQ ID NO: 17. In some embodiments, the one or more reporter genes further comprise a resistance marker, such as a neomycin resistance marker. In some embodiments, the neomycin resistance marker is encoded by the nucleic acid sequence of SEQ ID NO: 11. In some embodiments, the neomycin resistance marker comprises the peptide sequence of SEQ ID NO: 18. In some embodiments, the insulin resistance reporter comprises two or more reporter genes, and the two or more reporter genes are separated by one or more bicistronic elements. In some embodiments, the one or more bicistronic elements comprise one or more self-cleaving peptides or IRES. In some embodiments, the one or more self-cleaving peptides comprise a P2A, T2A, E2A, or F2A self-cleaving peptide. The separation of the two or more reporter genes by the one or more bicistronic elements allows for expression of a plurality of separate (i.e. not fused) reporter proteins. In some embodiments, the 5′ homology region associated with the insulin-dependent gene comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% homology to SEQ ID NO: 6. In some embodiments, the 3′ homology region associated with the insulin-dependent gene comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% homology to SEQ ID NO: 13. In some embodiments, the insulin resistance reporter comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 4. Any of the cells, tissues, or organoids, including liver organoids, can be genetically modified to expressed any of these insulin resistance reporters.

### Cell, Tissue and Organoid Compositions

For the methods disclosed herein, pluripotent stem cells (PSCs) can be used directly or differentiated into downstream cell types. In some embodiments, the PSCs are differentiated into definitive endoderm cells. In some embodiments, the PSCs are differentiated into anterior foregut cells. In some embodiments, the PSCs are differentiated into insulin responsive cells, tissues, or organoids. In some embodiments, the insulin responsive cells, tissues, or organoids are or comprise pancreatic cells, brain cells, adipose cells, muscle cells, or liver cells. In some embodiments, the insulin responsive cells, tissues, or organoids are liver organoids. In some embodiments, the PSCs are differentiated into a liver organoid. In some embodiments, the PSCs are differentiated into pancreatic cells, brain cells, adipose cells, muscle cells, or liver cells, or tissues or organoids thereof, by methods known in the art.

Disclosed herein are insulin responsive cells, tissues, or organoids comprising an insulin resistance reporter. In some embodiments, the insulin resistance reporter is any one of the insulin resistance reporters disclosed herein. In some embodiments, the insulin resistance reporter is operatively linked to an insulin-dependent gene of the insulin responsive cell, tissue, or organoid. In some embodiments, the insulin-dependent gene is a gluconeogenesis gene or a lipogenesis gene. In some embodiments, the insulin-dependent gene is selected from the group consisting of PCK1, G6PC, G6PC2, G6PC3, GSK3A, GSK3B, MTOR, GCK, FOXO1, CREB1, TFE1, TFE3, SREBP1C, FASN, ACLY, and ACC. In some embodiments, the insulin-dependent gene is PCK1. In some embodiments, expression of the insulin-dependent gene results in expression of the insulin resistance reporter. In some embodiments, the insulin-dependent gene and the insulin resistance reporter are separated by a bicistronic element. In some embodiments, the insulin resistance reporter is 3′ of the insulin-dependent gene, such that the insulin resistance reporter and the insulin-dependent gene are separated by a bicistronic element. In some embodiments, the insulin resistance reporter is 5′ of the insulin-dependent gene, such that the insulin resistance reporter and the insulin-dependent gene are separated by a bicistronic element. In some embodiments, the bicistronic element is a self-cleaving peptide or an IRES. In some embodiments, the self-cleaving peptide is a P2A, T2A, E2A, or F2A self-cleaving peptide. In some embodiments, the insulin resistance reporter has been integrated at the locus of the insulin-dependent gene using a CRISPR nuclease. In some embodiments, the CRISPR nuclease is Cas9. However, any other method of gene editing can be used to integrate the insulin resistance reporter to the locus of the insulin-dependent gene. In some embodiments, the insulin resistance reporter comprises one or more reporter genes. In some embodiments, the one or more reporter genes comprise a gene encoding for a fluorescent protein or a gene encoding for a luminescent protein, or both. In some embodiments, the fluorescent protein comprises mScarlet or the luminescent protein comprises luciferase. However, any other fluorescent protein and any other luminescent protein generally known in the art can be used. In some embodiments, mScarlet is encoded by the nucleic acid sequence of SEQ ID NO: 8. In some embodiments, mScarlet comprises the peptide sequence of SEQ ID NO: 15. In some embodiments, the one or more reporter genes further comprise a resistance marker. In some embodiments, luciferase is encoded by the nucleic acid sequence of SEQ ID NO: 10. In some embodiments, luciferase comprises the peptide sequence of SEQ ID NO: 17. In some embodiments, the one or more reporter genes further comprises a resistance marker. In some embodiments, the resistance marker is a neomycin resistance marker. In some embodiments, the neomycin resistance marker is encoded by the nucleic acid sequence of SEQ ID NO: 11. In some embodiments, the neomycin resistance marker comprises the peptide sequence of SEQ ID NO: 18. In some embodiments, the insulin resistance reporter comprises two or more reporter genes, and the two or more reporter genes are separated by one or more bicistronic elements. In some embodiments, the one or more bicistronic elements comprise one or more self-cleaving peptides or IRES. In some embodiments, the one or more self-cleaving peptides comprise a P2A, T2A, E2A, or F2A self-cleaving peptide. In some embodiments, the insulin resistance reporter comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 4. In some embodiments, the insulin responsive cell, tissue, or organoid is a stem cell, induced pluripotent stem cell, embryonic stem cell, definitive endoderm cell, or a foregut cell. In some embodiments, the insulin responsive cell, tissue, or organoid is a mammalian or human insulin responsive cell, tissue, or organoid. In some embodiments, the insulin responsive cell, tissue, or organoid has been derived from pluripotent stem cells, induced pluripotent stem cells, or embryonic stem cells. In some embodiments, the insulin responsive cell, tissue, or organoid has been derived from cells from a subject having or at risk of developing a disease or disorder associated with insulin dysfunction. In some embodiments, insulin dysfunction can comprise insulin resistance or insulin hypersensitivity. In some embodiments, the disease or disorder associated with insulin dysfunction comprises diabetes, metabolic syndrome, fatty liver disease, steatohepatitis, obesity, cardiovascular disease, polycystic ovary syndrome, hyperglycemia, hyperinsulinemia, dyslipidemia, or any combination thereof.

Methods of producing liver organoids have been explored previously in, for example, PCT Publications WO 2018/085615, WO 2018/085622, WO 2018/085623, WO 2018/191673, WO 2018/226267, WO 2019/126626, WO 2020/023245, and WO 2020/069285, each of which is hereby expressly incorporated by references in its entirety. Any known liver organoid composition or the methods of making thereof are applicable to the human liver organoids (HLOs) described herein.

Also disclosed herein are liver organoids comprising an insulin resistance reporter. In some embodiments, the insulin resistance reporter is any one of the insulin resistance reporters disclosed herein. In some embodiments, the insulin resistance reporter is operatively linked to an insulin-dependent gene. In some embodiments, the insulin-dependent gene is a gluconeogenesis gene or a lipogenesis gene. In some embodiments, the insulin-dependent gene is selected from the group consisting of PCK1, G6PC, G6PC2, G6PC3, GSK3A, GSK3B, MTOR, GCK, FOXO1, CREB1, TFE1, TFE3, SREBP1C, FASN, ACLY, and ACC. In some embodiments, the insulin-dependent gene is PCK1. In some embodiments, expression of the insulin-dependent gene results in expression of the insulin resistance reporter. In some embodiments, the insulin-dependent gene and the insulin resistance reporter are separated by a bicistronic element. In some embodiments, the insulin resistance reporter is 3′ of the insulin-dependent gene, such that the insulin resistance reporter and the insulin-dependent gene are separated by a bicistronic element. In some embodiments, the insulin resistance reporter is 5′ of the insulin-dependent gene, such that the insulin resistance reporter and the insulin-dependent gene are separated by a bicistronic element. In some embodiments, the bicistronic element is a self-cleaving peptide or an IRES. In some embodiments, the self-cleaving peptide is a P2A, T2A, E2A, or F2A self-cleaving peptide. In some embodiments, the insulin resistance reporter has been integrated at the locus of the insulin-dependent gene using a CRISPR nuclease. In some embodiments, the CRISPR nuclease is Cas9. However, any other method of gene editing can be used to integrate the insulin resistance reporter to the locus of the insulin-dependent gene. In some embodiments, the insulin resistance reporter comprises one or more reporter genes. In some embodiments, the one or more reporter genes comprise a gene encoding for a fluorescent protein or a gene encoding for a luminescent protein, or both. In some embodiments, the fluorescent protein comprises mScarlet or the luminescent protein comprises luciferase. However, any other fluorescent protein and any other luminescent protein generally known in the art can be used. In some embodiments, mScarlet is encoded by the nucleic acid sequence of SEQ ID NO: 8. In some embodiments, mScarlet comprises the peptide sequence of SEQ ID NO: 15. In some embodiments, the one or more reporter genes further comprise a resistance marker. In some embodiments, luciferase is encoded by the nucleic acid sequence of SEQ ID NO: 10. In some embodiments, luciferase comprises the peptide sequence of SEQ ID NO: 17. In some embodiments, the one or more reporter genes further comprises a resistance marker. In some embodiments, the resistance marker is a neomycin resistance marker. In some embodiments, the neomycin resistance marker is encoded by the nucleic acid sequence of SEQ ID NO: 11. In some embodiments, the neomycin resistance marker comprises the peptide sequence of SEQ ID NO: 18. In some embodiments, the insulin resistance reporter comprises two or more reporter genes, and the two or more reporter genes are separated by one or more bicistronic elements. In some embodiments, the one or more bicistronic elements comprise one or more self-cleaving peptides or IRES. In some embodiments, the one or more self-cleaving peptides comprise a P2A, T2A, E2A, or F2A self-cleaving peptide. In some embodiments, the insulin resistance reporter comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 4. In some embodiments, the liver organoid is a fatty liver organoid or steatohepatitis liver organoid. In some embodiments, the fatty liver organoid or steatohepatitis liver organoid comprises a large number of fat droplets compared to a normal liver organoid. In some embodiments, the fatty liver organoid or steatohepatitis liver organoid is generated by contacting the liver organoid with fatty acids. In some embodiments, the fatty acids comprise oleic acid, linoleic acid, palmitic acid, or stearic acid, or any combination thereof. In some embodiments, the fatty liver organoid or steatohepatitis liver organoid exhibits insulin resistance and/or a type 2 diabetic phenotype. In some embodiments, insulin resistance comprises decreased AKT phosphorylation, reduced suppression of expression of PCK1, CREB1, or FOXO1 in response to insulin, or reduced suppression of gluconeogenesis in response to insulin, or any combination thereof, relative to a normal liver organoid. In some embodiments, the fatty liver organoid or steatohepatitis liver organoid exhibits more fat droplets, increased expression of DGAT½, or increased expression and/or secretion of pro-inflammatory cytokines, or any combination thereof, relative to a normal liver organoid. In some embodiments, the pro-inflammatory cytokines comprise TNFa, TGFb, IL6, IL8, or IL1b, or any combination thereof. In some embodiments, the liver organoid is a mammalian or human liver organoid. In some embodiments, the liver organoid has been derived from pluripotent stem cells, induced pluripotent stem cells, or embryonic stem cells. In some embodiments, the liver organoid has been derived from cells from a subject having or at risk of developing a disease or disorder associated with insulin dysfunction. In some embodiments, insulin dysfunction can comprise insulin resistance or insulin hypersensitivity. In some embodiments, the disease or disorder associated with insulin dysfunction comprises diabetes, metabolic syndrome, fatty liver disease, steatohepatitis, obesity, cardiovascular disease, polycystic ovary syndrome, hyperglycemia, hyperinsulinemia, dyslipidemia, or any combination thereof.

In some embodiments, the insulin responsive cells, tissues, or organoids are treated with a compound or composition to induce a lipidemic phenotype. In some embodiments, the compound or composition comprises one or more fatty acids. In some embodiments, the one or more fatty acids comprise oleic acid, linoleic acid, palmitic acid, or any combination thereof. In some embodiments, the insulin responsive cells, tissues, or organoids are liver organoids. In some embodiments, the liver organoids treated with the compound or composition to induce a lipidemic phenotype causes a fatty liver phenotype. In some embodiments, the liver organoids with a fatty liver phenotype are fatty liver organoids. In some embodiments, the fatty liver organoids resemble liver tissue exhibiting nonalcoholic fatty liver disease (NAFLD). In some embodiments, the fatty liver organoids resemble liver tissue exhibiting steatohepatitis (i.e. steatohepatitis liver organoids). In some embodiments, the insulin responsive cells, tissues, or organoids exhibit insulin resistance after treatment with the compound or composition used to induce the lipidemic phenotype. Methods of generating fatty liver organoids and/or steatohepatitis organoids using fatty acids are explored in PCT Publication WO 2018/085622, which is hereby expressly incorporated by reference in its entirety. In some embodiments, the lipidemic phenotype is fully or partially reversed by treatment of the insulin responsive cells, tissues, or organoids with one or more (e.g. at least 1, 2, 3) of obeticholic acid, pioglitzone, or metformin, or any combination thereof. Use of obeticholic acid for the treatment of fatty liver disease has been explored in PCT Publication WO 2018/085623, hereby expressly incorporated by reference in its entirety.

Also disclosed herein are stem cells comprising an insulin resistance reporter. In some embodiments, the insulin resistance reporter is any one of the insulin resistance reporters disclosed herein. In some embodiments, the stem cell is an induced pluripotent stem cell (iPSC) or embryonic stem cell (ESC). In some embodiments, the stem cell is an iPSC. In some embodiments, the stem cell is derived from a human subject. In some embodiments, the insulin resistance reporter comprises one or more nucleic acid sequences encoding for a reporter protein and one or more nucleic acid sequences encoding for a self-cleaving peptide separating each of the one or more nucleic acid sequence encoding for the reporter protein. In some embodiments, the insulin resistance reporter comprises one or more nucleic acid sequences having at least 80%, 85%, 90%, 91%, 92%, 93%, 94%, 95%, 96%, 97%, 98%, or 99% homology to a sequence encoding for a reporter protein and one or more nucleic acid sequences having at least 80%, 85%, 90%, 91%, 92%, 93%, 94%, 95%, 96%, 97%, 98%, or 99% homology to a sequence encoding for a self-cleaving peptide separating each of the sequences encoding for the reporter protein. In some embodiments, the reporter protein is a fluorescent protein. In some embodiments, the fluorescent protein is mScarlet, or any other fluorescent protein known in the art. In some embodiments, mScarlet is encoded by the nucleic acid sequence of SEQ ID NO: 8. In some embodiments, mScarlet comprises the peptide sequence of SEQ ID NO: 15. In some embodiments, the reporter protein is a luminescent protein. In some embodiments, the luminescent protein is luciferase, or any other luminescent protein known in the art. In some embodiments, luciferase is encoded by the nucleic acid sequence of SEQ ID NO: 10. In some embodiments, luciferase comprises the peptide sequence of SEQ ID NO: 17. In some embodiments, the reporter protein further comprises a resistance marker. In some embodiments, the resistance marker is a neomycin resistance marker. In some embodiments, the neomycin resistance marker is encoded by the nucleic acid sequence of SEQ ID NO: 11. In some embodiments, the neomycin resistance marker comprises the peptide sequence of SEQ ID NO: 18. In some embodiments, the insulin resistance reporter further comprises one or more nucleic acid sequences having at least 80%, 85%, 90%, 91%, 92%, 93%, 94%, 95%, 96%, 97%, 98%, 99%, or 100% homology to a sequence associated with an insulin-dependent gene. In some embodiments, the insulin-dependent gene is a gluconeogenesis gene or a lipogenesis gene. In some embodiments, the insulin-dependent gene is selected from the group consisting of PCK1, G6PC, G6PC2, G6PC3, GSK3A, GSK3B, MTOR, GCK, FOXO1, CREB1, TFE1, TFE3, SREBP1C, FASN, ACLY, and ACC. In some embodiments, the insulin-dependent gene is PCK1. In some embodiments, the one or more nucleic acid sequences having homology to the sequence associated with the insulin-dependent gene are flanking the sequences encoding for the reporter protein and the sequences encoding for the self-cleaving peptide, wherein the sequences having homology to the sequence associated with the insulin-dependent gene act as homology regions for recombination into the genome of the liver organoid. In some embodiments, the insulin resistance reporter has been integrated into the genome of the stem cell using a CRISPR nuclease. In some embodiments, the CRISPR nuclease is Cas9. However, any other method of gene editing known in the art can be used to integrate the insulin resistance reporter into the genome of the stem cell. In some embodiments, the sequence associated with an insulin-dependent gene comprises a 5′ homology region and a 3′ homology region associated with the insulin-dependent gene. The 5′ homology region and 3′ homology region permits integration of the insulin resistance reporter to the locus of the insulin-dependent gene by homology directed repair. In some embodiments, the 5′ homology region associated with the insulin-dependent gene comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% homology to SEQ ID NO: 6; and/or wherein the 3′ homology region associated with the insulin-dependent gene comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% homology to SEQ ID NO: 13. In some embodiments, the insulin resistance reporter comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 4.

Also disclosed herein are definitive endoderm cells differentiated from any of the stem cells disclosed herein. Also disclosed herein are anterior foregut cells differentiated from any one of the stem cells disclosed herein. Also disclosed herein are insulin responsive cells, tissues, or organoids differentiated from any of the stem cells disclosed herein. In some embodiments, the insulin responsive cells, tissues, or organoids comprises pancreatic cells, brain cells, adipose cells, muscle cells, or liver cells. In some embodiments, the insulin responsive cell, tissue, or organoid is a liver organoid. In some embodiments, the liver organoid is a fatty liver organoid after treatment of the liver organoid with one or more fatty acids. In some embodiments, the one or more fatty acids comprise oleic acid, linoleic acid, palmitic acid, or any combination thereof. Also disclosed herein are pancreatic cells, brain cells, adipose cells, muscle cells, or liver cells differentiated from any of the stem cells disclosed herein. In some embodiments, any of the cells differentiated from any of the stem cells disclosed herein comprise any one of the insulin resistance reporters disclosed herein. In some embodiments, any of the cells disclosed herein exhibits insulin dysfunction. In some embodiments, any of the cells disclosed herein exhibits insulin resistance.

In some embodiments, any of the cells disclosed herein may be cryopreserved for later use. The cells can be cryopreserved according to methods generally known in the art.

### Methods of Screening, Methods of Use as Indicators, and Pharmaceutical Compositions

Disclosed herein are in vitro methods of screening for candidate compounds for the treatment of a disease or disorder associated with insulin dysfunction. In some embodiments, the methods comprise contacting a liver organoid comprising an insulin resistance reporter or an insulin responsive cell, tissue, or organoid comprising an insulin resistance reporter with the candidate compounds and observing an improvement in the disease or disorder associated with insulin dysfunction in the liver organoid or the insulin responsive cell, tissue, or organoid. In some embodiments, the disease or disorder associated with insulin dysfunction comprises diabetes, metabolic syndrome, fatty liver disease, steatohepatitis, obesity, cardiovascular disease, polycystic ovary syndrome, hyperglycemia, hyperinsulinemia, dyslipidemia, or any combination thereof. In some embodiments, the liver organoid comprising the insulin resistance reporter is any one of the liver organoids disclosed herein. In some embodiments, the insulin responsive cell, tissue, or organoid comprising the insulin resistance reporter is any one of the insulin responsive cells, tissues, or organoids disclosed herein. In some embodiments, the liver organoid comprising the insulin resistance reporter is a fatty liver organoid or a steatohepatitis liver organoid. In some embodiments, observing an improvement in the disease or disorder associated with insulin dysfunction in the liver organoid comprises observing increased AKT phosphorylation, increased suppression of expression of PCK1, CREB1, or FOXO1 in response to insulin, or increased suppression of gluconeogenesis in response to insulin, or any combination thereof, relative to before the contacting step. In some embodiments, observing an improvement in the disease or disorder associated with insulin dysfunction in the liver organoid comprises observing a decreased number of fat droplets, reduced expression of DGAT½, or decreased expression and/or secretion of pro-inflammatory cytokines, or any combination thereof, relative to before the contacting step. In some embodiments, the insulin resistance reporter is 3′ of the insulin-dependent gene of the liver organoid or the insulin responsive cell, tissue, or organoid, such that the insulin resistance reporter and the insulin-dependent gene are separated by a bicistronic element. In some embodiments, the insulin resistance reporter is 5′ of the insulin-dependent gene of the liver organoid or the insulin responsive cell, tissue, or organoid, such that the insulin resistance reporter and the insulin-dependent gene are separated by a bicistronic element.

Also disclosed herein are in vitro methods of assessing insulin resistance of an insulin responsive cell, tissue, or organoid comprising an insulin resistance reporter. In some embodiments, the insulin resistance reporter is any one of the insulin resistance reporters disclosed herein. In some embodiments, the methods comprise quantifying a baseline expression level of one or more reporter proteins of the insulin resistance reporter, contacting the insulin responsive cell, tissue, or organoid with insulin or a derivative or mimetic thereof, quantifying a post-treatment expression level of the one or more reporter proteins, and determining based on the change in expression level or lack thereof of the one or more reporter proteins that the insulin responsive cell, tissue, or organoid exhibits insulin resistance. In some embodiments, the methods further comprise contacting the insulin responsive cell, tissue, or organoid with one or more of obeticholic acid (OCA), pioglitazone, or metformin, or any combination thereof. In some embodiments, the methods further comprise contacting the insulin responsive cell, tissue, or organoid with one or more fatty acids prior to quantifying the baseline expression level. In some embodiments, the methods further comprise contacting the insulin responsive cell, tissue, or organoid with one or more fatty acids after quantifying the baseline expression level and prior to contacting the insulin responsive cell, tissue, or organoid with insulin or the derivative or mimetic thereof. In some embodiments, the insulin responsive cell, tissue, or organoid is any one of the insulin responsive cells, tissues, or organoids disclosed herein. In some embodiments, the one or more fatty acids comprise oleic acid, linoleic acid, palmitic acid, or any combination thereof. In some embodiments, the insulin responsive cell, tissue, or organoid is derived from a human subject in need of treatment for insulin resistance. In some embodiments, the insulin responsive cell, tissue, or organoid is a liver organoid and the insulin resistance is hepatic insulin resistance. In some embodiments, the hepatic insulin resistance is caused by nonalcoholic fatty liver disease (NAFLD) or nonalcoholic steatohepatitis (NASH). In some embodiments, the insulin resistance reporter is 3′ of the insulin-dependent gene of the liver organoid or the insulin responsive cell, tissue, or organoid, such that the insulin resistance reporter and the insulin-dependent gene are separated by a bicistronic element. In some embodiments, the insulin resistance reporter is 5′ of the insulin-dependent gene of the liver organoid or the insulin responsive cell, tissue, or organoid, such that the insulin resistance reporter and the insulin-dependent gene are separated by a bicistronic element.

Also disclosed herein are in vitro methods of screening for a compound or composition that treats insulin resistance. In some embodiments, the methods comprise contacting an insulin responsive cell, tissue, or organoid comprising an insulin resistance reporter with one or more fatty acids, quantifying a baseline expression level of one or more reporter proteins of the insulin resistance reporter, contacting the insulin responsive cell, tissue, or organoid with the compound or composition, quantifying a post-treatment expression level of the one or more reporter proteins, and determining based on the change in expression level or lack thereof of the one or more reporter proteins that the compound or composition can treat the insulin resistance. In some embodiments, the insulin responsive cell, tissue, or organoid is any one of the insulin responsive cells, tissues, or organoids disclosed herein. In some embodiments, the insulin resistance reporter is any one of the insulin resistance reporters disclosed herein. In some embodiments, the one or more fatty acids comprise oleic acid, linoleic acid, palmitic acid, or any combination thereof. In some embodiments, the insulin responsive cell, tissue, or organoid is derived from a human subject in need of treatment for insulin resistance. In some embodiments, the insulin responsive cell, tissue, or organoid is a liver organoid and the insulin resistance is hepatic insulin resistance. In some embodiments, the hepatic insulin resistance is caused by nonalcoholic fatty liver disease (NAFLD) or nonalcoholic steatohepatitis (NASH). In some embodiments, the insulin resistance reporter is 3′ of the insulin-dependent gene of the liver organoid or the insulin responsive cell, tissue, or organoid, such that the insulin resistance reporter and the insulin-dependent gene are separated by a bicistronic element. In some embodiments, the insulin resistance reporter is 5′ of the insulin-dependent gene of the liver organoid or the insulin responsive cell, tissue, or organoid, such that the insulin resistance reporter and the insulin-dependent gene are separated by a bicistronic element.

Also disclosed herein are the compounds or compositions identified by any of the screening methods disclosed herein that are found to have an effect towards treating, ameliorating, or preventing insulin resistance, or a disease or disorder associated with insulin dysfunction.

Some embodiments described herein relate to pharmaceutical compositions that comprise, consist essentially of, or consist of an effective amount of any one of the insulin responsive cells, tissues, or organoids described herein and a pharmaceutically acceptable carrier, excipient, or combination thereof. A pharmaceutical composition described herein is suitable for human and/or veterinary applications. In some embodiments, the insulin responsive cells, tissues or organoids (such as liver organoids) comprising an insulin resistance reporter can be used as a detection device to measure the levels of circulating insulin in a subject.

Also disclosed herein are methods of monitoring insulin response in a subject. In some embodiments, the methods comprise transplanting a liver organoid comprising an insulin resistance reporter or an insulin responsive cell, tissue, or organoid comprising an insulin resistance reporter to the subject and monitoring expression of the insulin resistance reporter of the liver organoid or the insulin responsive cell, tissue, or organoid. In some embodiments, the liver organoid comprising the insulin resistance reporter is any one of the liver organoids disclosed herein. In some embodiments, the insulin responsive cell, tissue, or organoid is any one of the insulin responsive cells, tissues, or organoids disclosed herein. In some embodiments, the subject has or is at risk of developing a disease or disorder associated with insulin dysfunction. In some embodiments, insulin dysfunction can comprise insulin resistance or insulin hypersensitivity. In some embodiments, the subject does not have or is not at risk of developing a disease or disorder associated with insulin dysfunction, and normal, physiological activity of insulin is monitored.

### EXAMPLES

### Example 1. Human Liver Organoids (HLOs) Can Be Used as a Model for Insulin Response

Liver organoids, such as those derived from human cells, can be produced according to the methods described herein and otherwise known in the art. For example, exemplary methods of producing liver organoids from pluripotent stem cells have been described in PCT Publications WO 2018/085615, WO 2018/191673, WO 2018/226267, WO 2019/126626, WO 2020/023245, and WO 2020/069285, each of which is hereby expressly incorporated by references in its entirety.

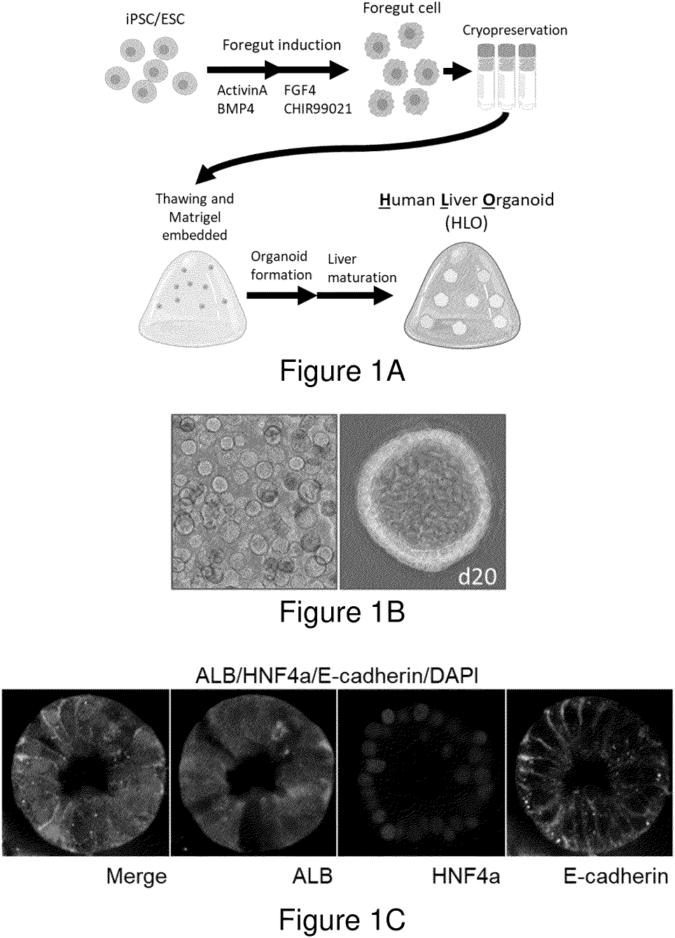

An embodiment of a schematic for the production of liver organoids is depicted in FIG. 1A. Briefly, pluripotent stem cells (e.g. iPSCs or ESCs) are first differentiated into definitive endoderm cells by culturing with Activin A and BMP4, and the definitive endoderm cells are subsequently cultured in a foregut induction medium comprising an FGF pathway activator (e.g. FGF4) and a Wnt pathway activator, which may be a GSK3 inhibitor (e.g. CHIR99021) to generate foregut cells, which may be in the form of spheroids. Optionally, these foregut cells may be cryopreserved for later use. The foregut cells can be embedded in a basement membrane matrix (e.g. Matrigel) and cultured with a differentiation medium comprising FGF2, VEGF, EGF, CHIR99021, and a TGF-b inhibitor to produce liver organoids. The resultant liver organoids have uniform morphology (FIG. 1B) and closely resemble liver tissue, expressing albumin (ALB) and hepatocyte nuclear factor 4 (HNF4), which are liver-specific markers, and the epithelial cell marker E-cadherin (FIG. 1C).

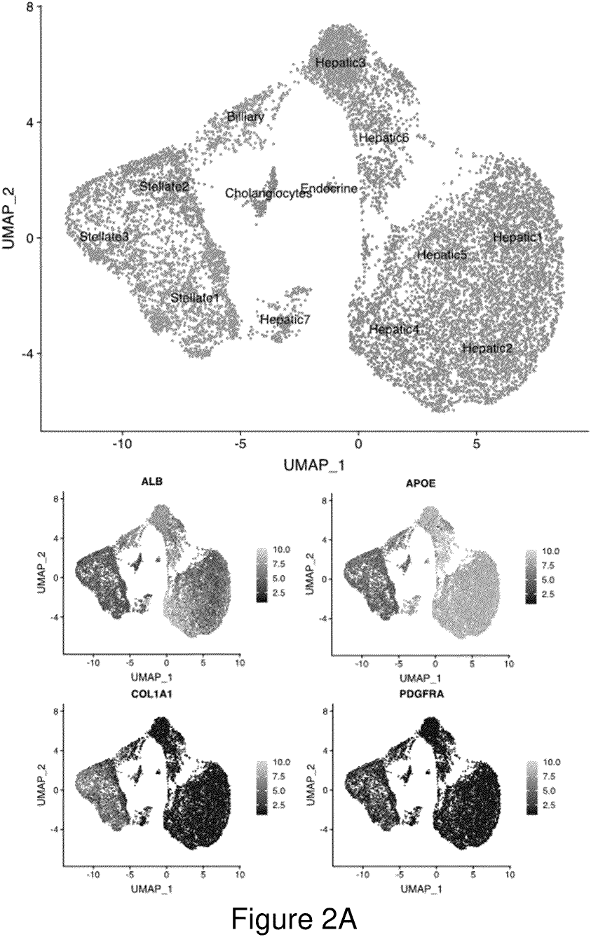

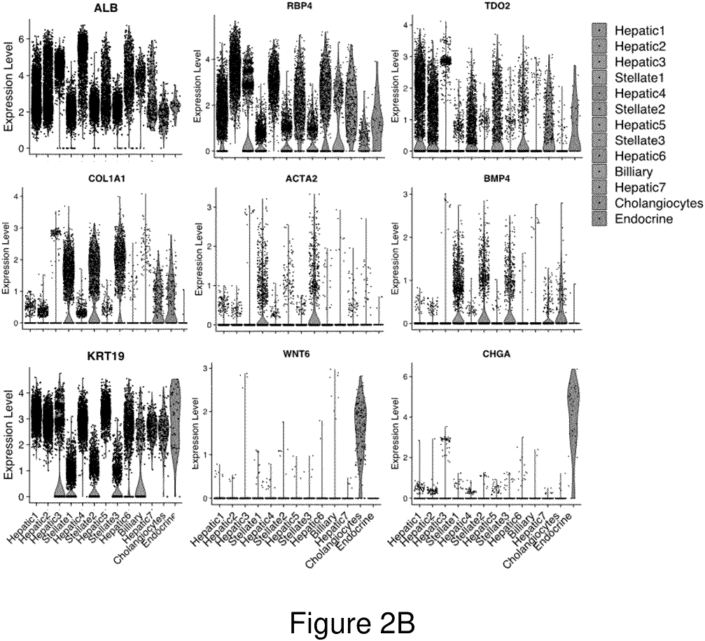

Single cell RNA sequencing (FIG. 2A) of the constituent cells of the liver organoids revealed distinct populations of 1) parenchymal cells (~82.4%) which express characteristic markers of hepatocytes, such as ALB, APOE, RBP4, and TDO2, and 2) non-parenchymal cells (~17.5%) which express characteristic markers of hepatic stellate cells, biliary cells, and cholangiocytes (COL1A1, PDGFRA, ACTA2, BMP4, WNT6) (FIG. 2B). These pluripotent stem cell-derived liver organoids have the potential to emulate the behavior of human liver tissue.

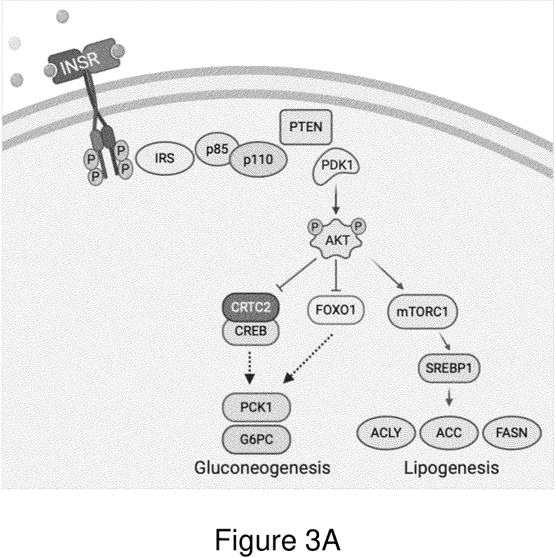

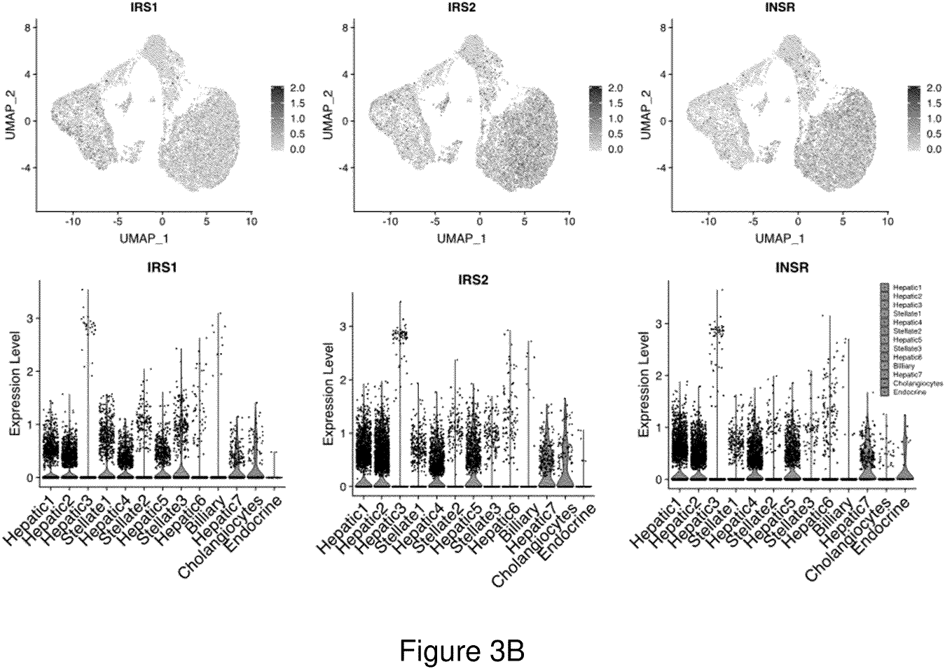

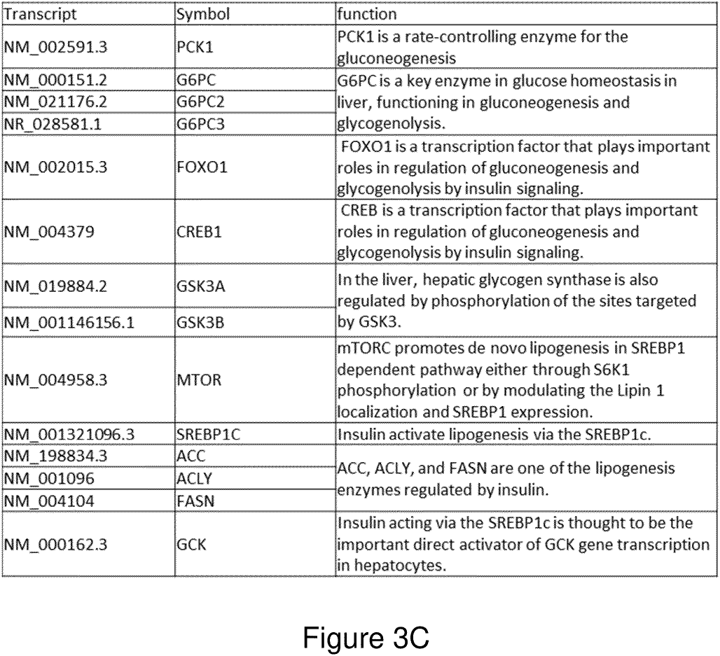

HLOs exhibit genes and pathways involved in insulin response. FIG. 3A depicts a schematic representation of the hepatic insulin response. Profiling of single cell RNA sequences of HLOs revealed that insulin receptor (INSR) and insulin receptor substrate 1 and 2 (IRS½) were expressed. INSR and IRS2 was concentrated in the hepatocyte population of the multicomponent HLOs, whereas IRS1 was also expressed in the stellate cell population.

HLOs showed a response to insulin stimulation of AKT phosphorylation, gluconeogenesis, and lipogenesis in vitro. Insulin responsiveness of HLOs was analyzed using Western blotting and qPCR. The prepared HLOs were cultured under insulin starvation for 24 hours and then exposed to insulin.

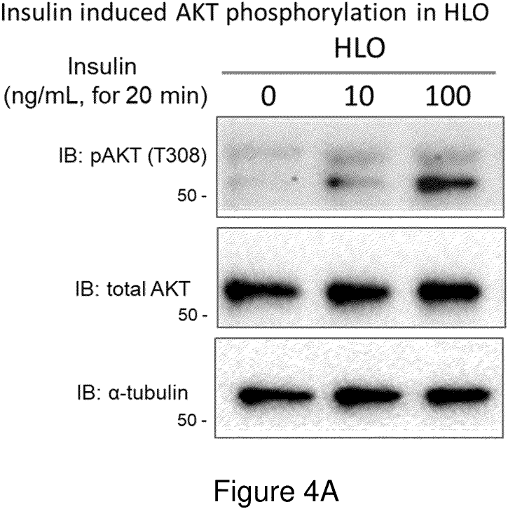

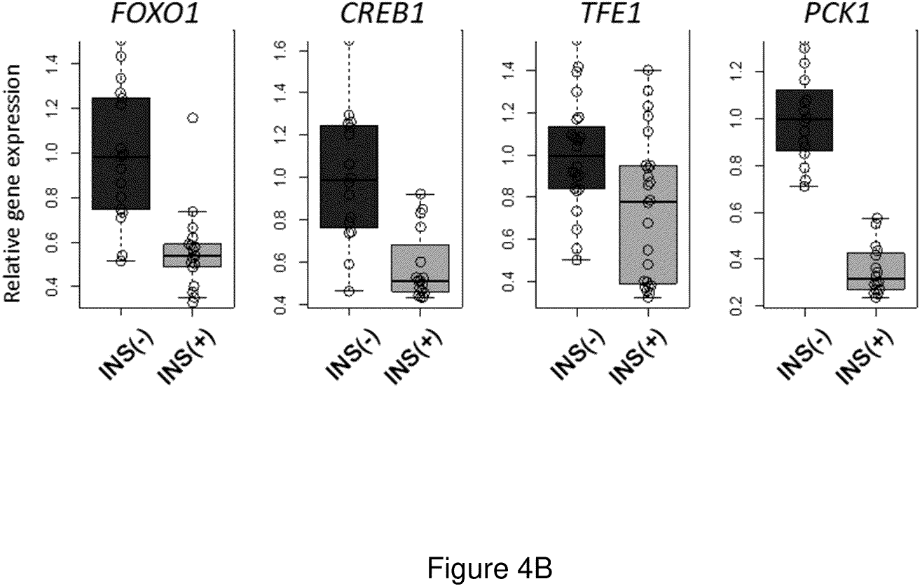

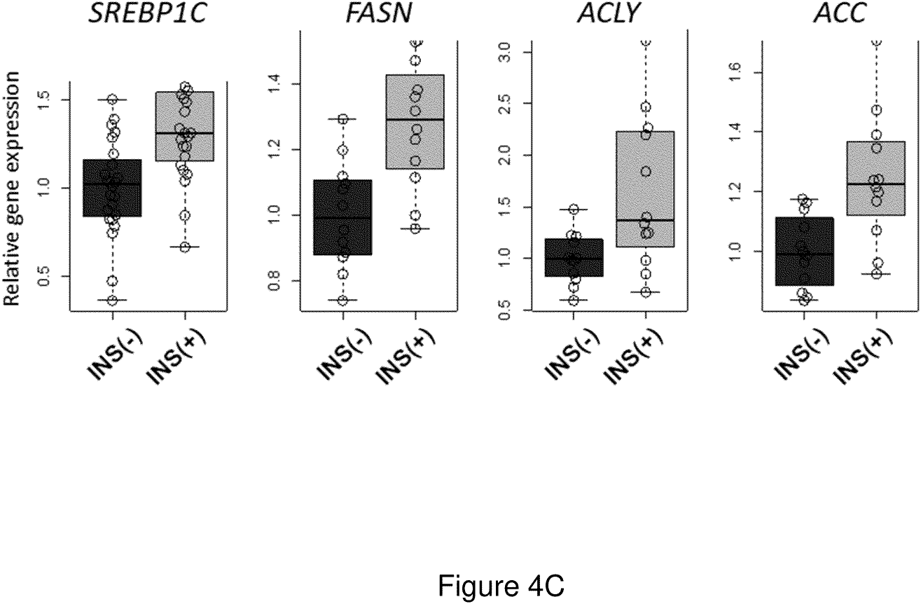

To analyze phosphorylation of AKT activated downstream of insulin signaling. HLOs were treated with insulin at 0 ng/mL, 10 ng/mL and 100 ng/mL for 20 minutes, and then proteins were extracted for Western blot. Insulin induces AKT phosphorylation in HLOs (FIG. 4A).

To analyze insulin responsiveness of gluconeogenesis and lipogenesis regulator genes in HLOs, they were treated with insulin at 100 ng/mL for 8 hours, and then RNA was extracted for qPCR. In the insulin-treated HLOs, gluconeogenesis regulatory gene expression (FOXO1, CREB1, TFE1, PCK1) was suppressed by insulin stimulation (FIG. 4B). Conversely, lipogenesis regulatory gene expression (SREBP, FASN, ACLY, ACC) was induced by insulin stimulation (FIG. 4C).

### Example 2. An Insulin Response Reporter Can Be Established in iPSCs and Downstream Cells

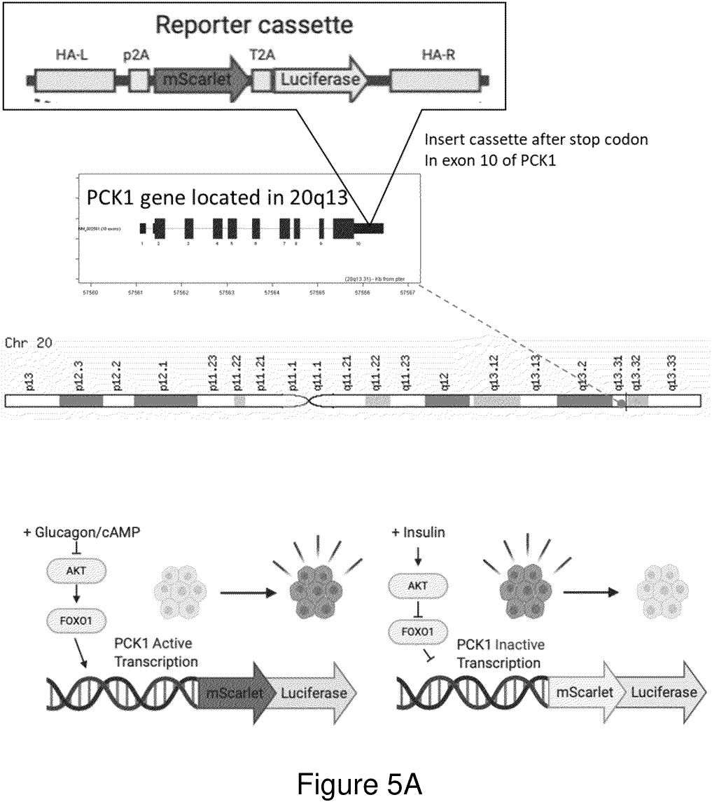

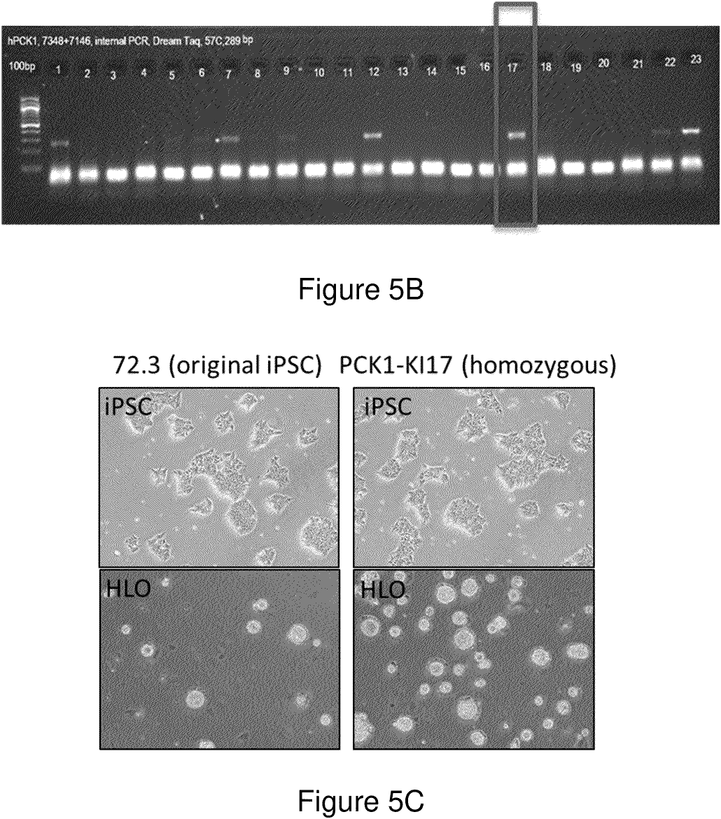

To visualize and quantify HLO insulin responsiveness, iPSCs were genetically edited using the CRISPR/Cas9 system to insert a reporter construct comprising mScarlet (fluorescence) and luciferase (luminescence) genes downstream of the gluconeogenesis regulatory gene PCK1, at the 3′ end in exon 10 (FIG. 5A). FIG. 5A also depicts a schematic of the reporter function. When gluconeogenesis is enhanced by exposure to glucagon or cAMP, mScarlet and luciferase are expressed together with increased expression of PCK1. When gluconeogenesis is suppressed by exposure to insulin, the expression of mScarlet and luciferase also decreases in correlation with the downregulation of PCK1. The exemplary reporter construct is represented as the nucleic acid sequence of SEQ ID NO: 4 and the peptide sequence of SEQ ID NO: 5. However, it is envisioned that other reporter constructs, such as those using alternative reporters, can be used. Furthermore, alternative insulin pathway regulatory genes are depicted in FIG. 3C to be used in lieu of PCK1. Exemplary sgRNAs for directing Cas9 to the 3′ of PCK1 are provided as SEQ ID NO: 1-3.

### Example 3. Insulin Responsiveness Can Be Observed in the PCK1 Reporter HLOs

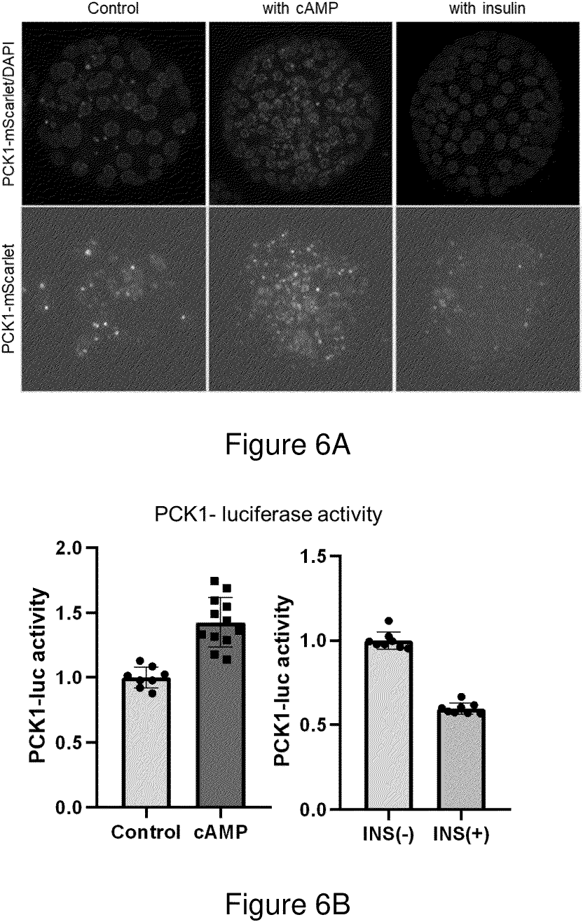

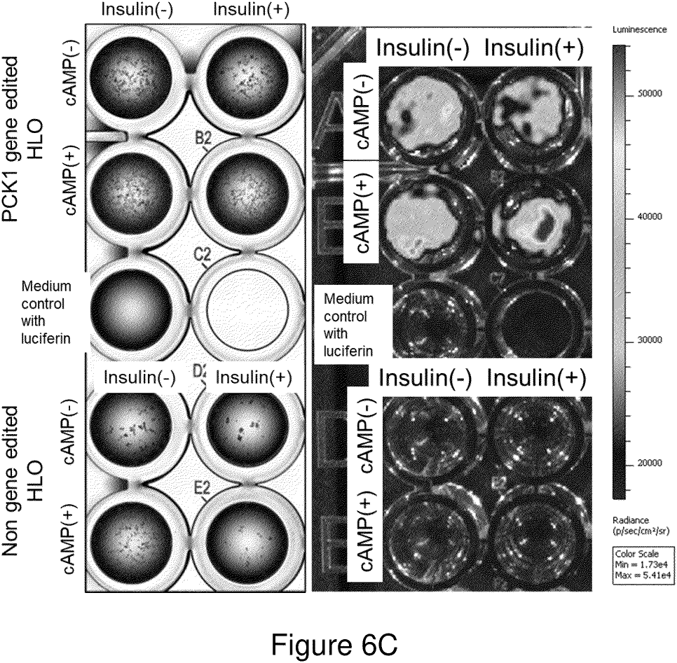

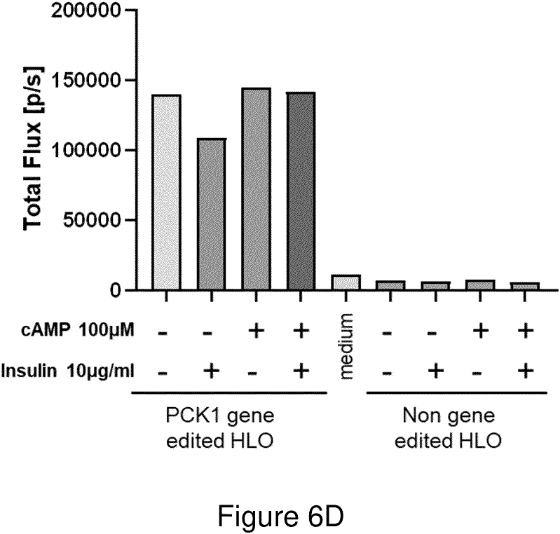

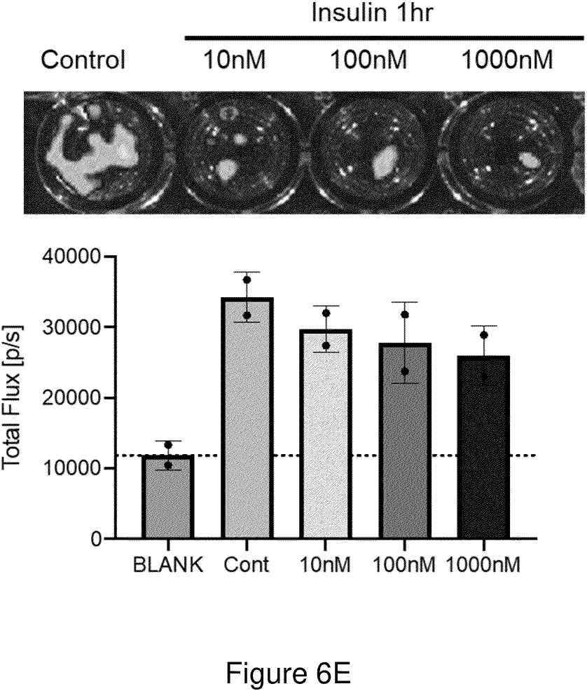

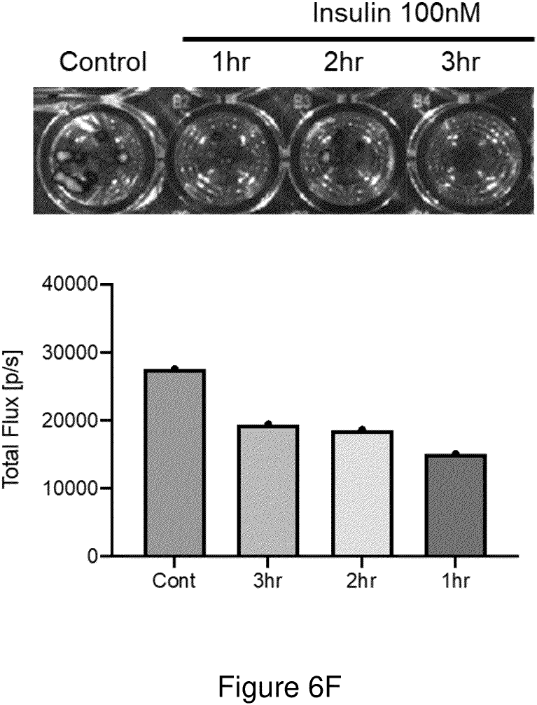

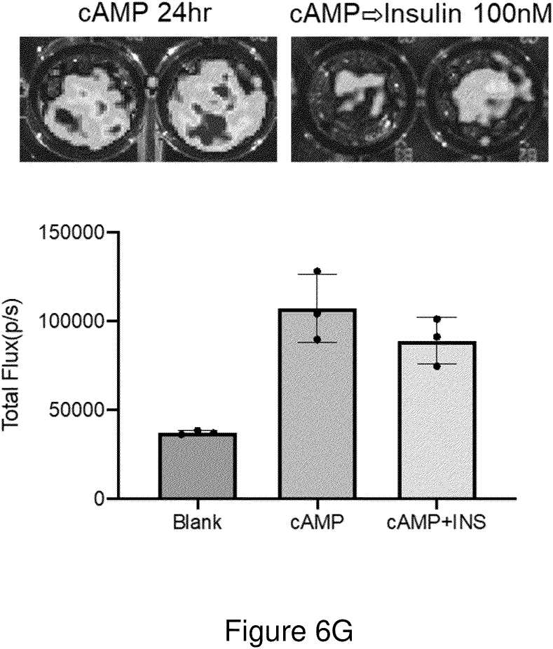

Human iPSCs were gene edited to insert the fluorescence and luminescence reporter construct to the 3′ end of PCK1. Insertion of the construct at the desired location was verified by amplification of the gene locus (FIG. 5B), and normal morphology was observed (FIG. 5C). These gene edited iPSCs were subsequently differentiated into HLOs. To analyze their insulin responsiveness, the HLOs were treated with cAMP at 100 µM for 24 hours. Subsequently, 100 ng/mL of insulin was applied for 3 hours, and fluorescence and luciferase activity were measured. As a result of cAMP treatment, PCK1-mScarlet fluorescence and luciferase activity were increased in response to gluconeogenesis (FIGS. 6A-B). In addition, as a result of treatment with 100 ng/mL insulin for 3 hours after treatment with cAMP, PCK1-mScarlet fluorescence and luciferase activity of PCK1 was decreased in response to insulin stimulation. FIGS. 6C-D depicts the detection of PCK1-luciferase luminescence as measured by in vitro imaging. HLOs derived from iPSCs that have not been gene edited with the reporter construct did not show any luciferase signal in any of the conditions (cAMP, insulin).

Various parameters of insulin response were tested with the reporter HLOs.

The effect of insulin concentration was tested. PCK1-luciferase HLOs were starved of insulin for 24 hours and then treated with 0 nM, 10 nM, 100 nM, or 1000 nM of insulin for 1 hour for luciferase imaging. The luciferase activity of PCK1 was decreased in response to insulin stimulation (FIG. 6E).

The effect of insulin treatment time was tested. Insulin starved HLOs were treated with 100 nM of insulin for 1, 2, or 3 hours (with no insulin control). Luciferase activity of PCK1 was reduced after 1 hour of treatment (FIG. 6F).

The effect of cAMP treatment was confirmed. Insulin starved HLOs were treated with 100 µM of cAMP for 24 hours, and then luciferase imaging was performed. In another condition, cAMP-treated HLOs were treated with 100 nM of insulin for 3 hours. PCK1-luciferase activity was increased by cAMP treatment and inhibited by insulin stimulation (FIG. 6G).

### Example 4. A Steatohepatitis Human Liver Organoid (sHLO) Model Exhibits Symptoms of Type 2 Diabetes

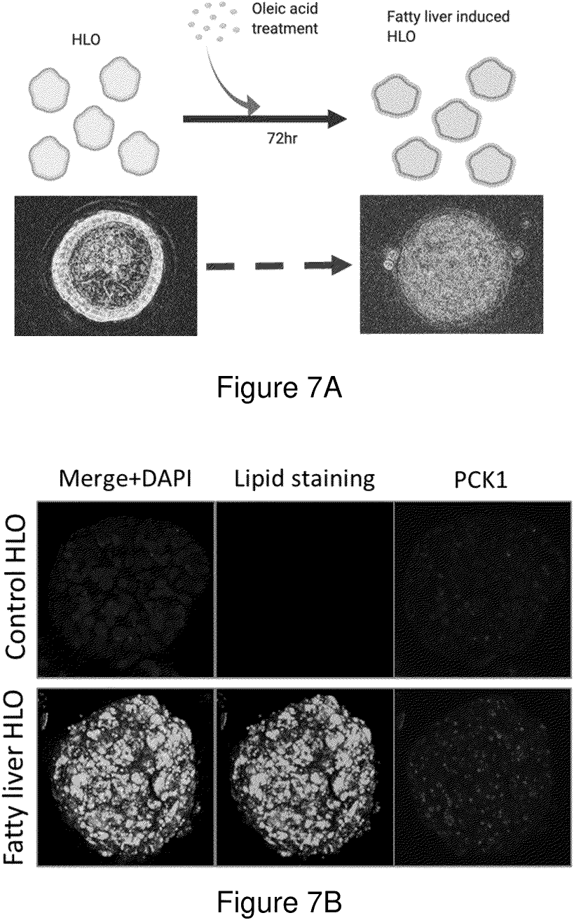

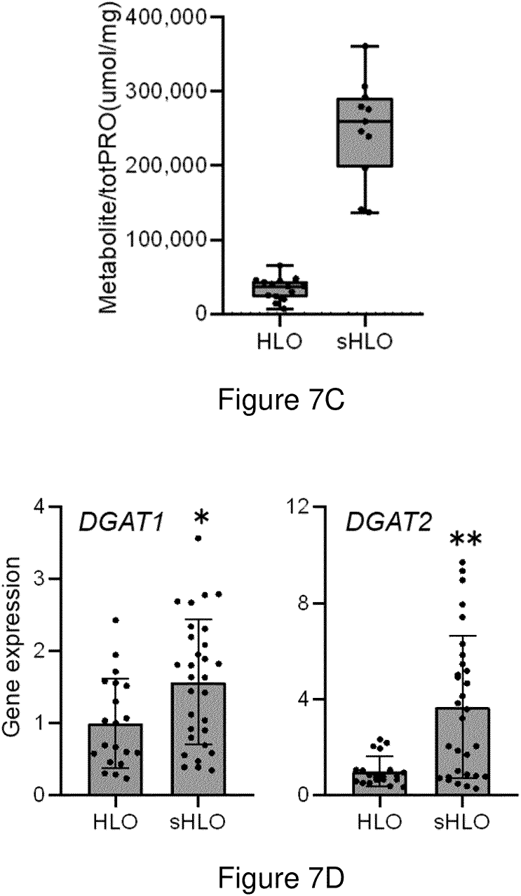

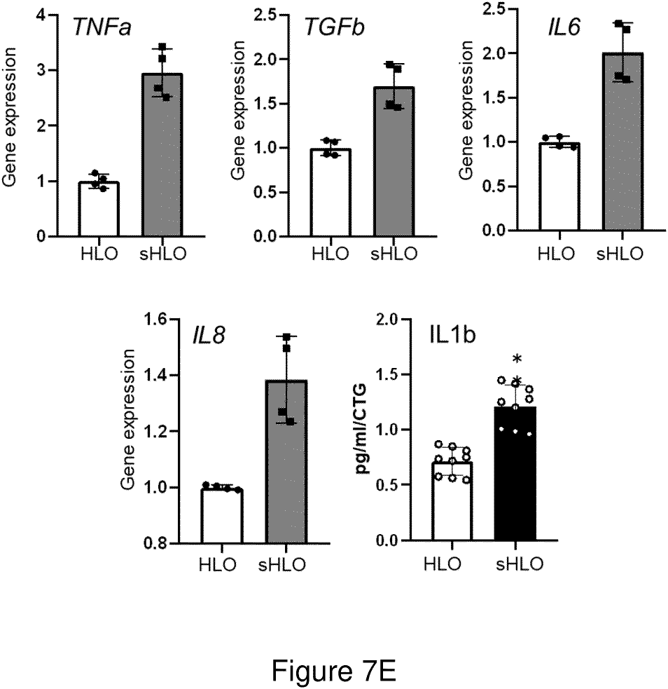

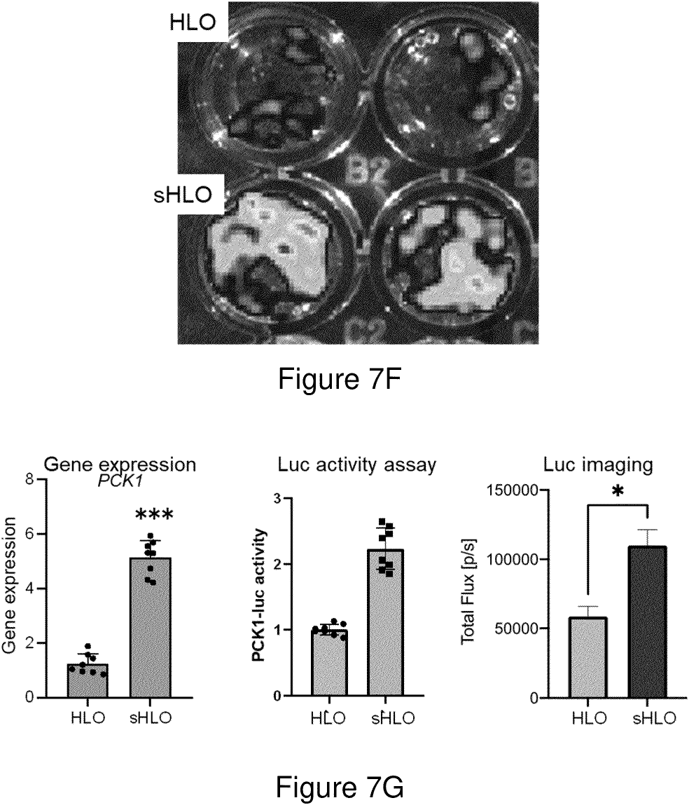

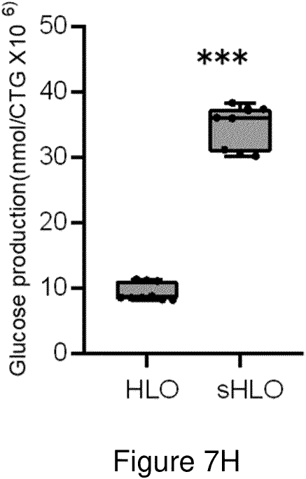

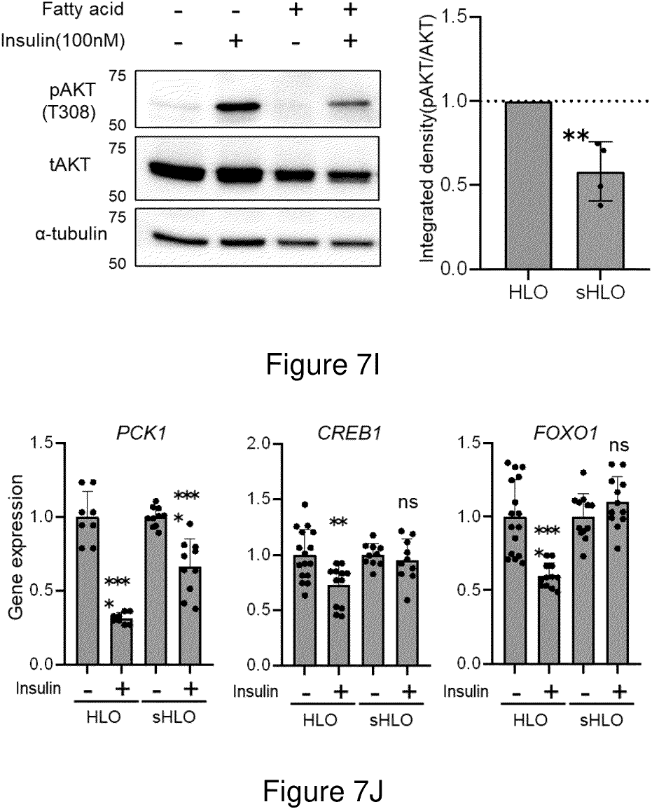

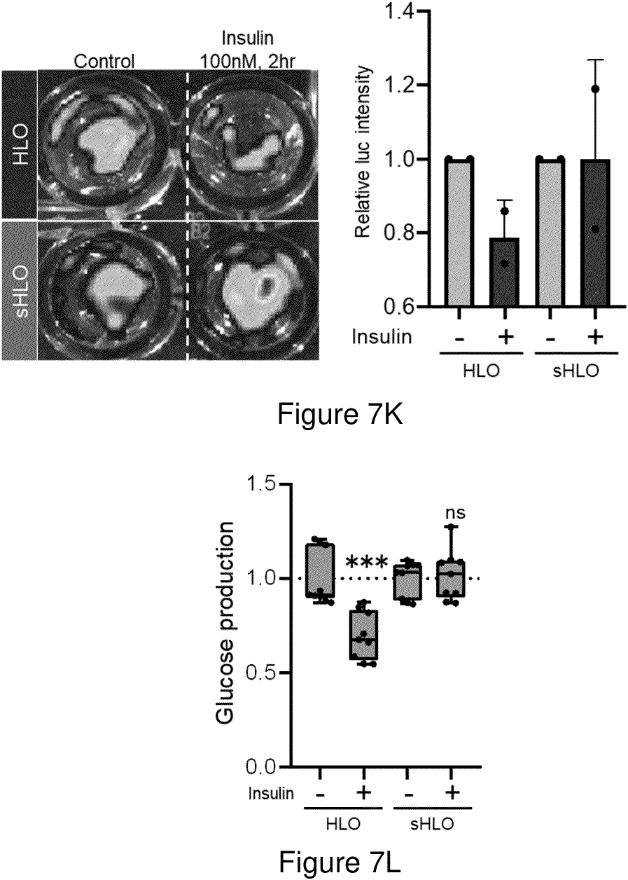

Fatty acid treatment induces a steatohepatitis phenotype in HLOs. Exposure to HLO to 300 µM of oleic acid for 72 hours induces fat accumulation (FIG. 7A). Fat imaging and NMR based analysis showed a large number of fat droplets in sHLO (FIGS. 7B-C). DGAT½, which catalyzes the formation of triglycerides from diacylglycerol and acyl-CoA, was increased in sHLO relative to normal HLOs (FIG. 7D). The expression and secretion of pro-inflammatory cytokines were also increased in sHLO (FIG. 7E).

sHLO showed excessive gluconeogenesis via constant activation of PCK1, which is a hallmark of type 2 diabetes. A marked increase in PCK1 was observed in sHLO induced by excessive fat accumulation, which was also accompanied by enhanced glucose production (FIGS. 7F-H).

Insulin responsiveness in the sHLO was examined. To detect insulin responsiveness, insulin starvation of sHLO and HLO controls was performed for 24 hours, followed by insulin stimulation.

Insulin signal analysis by Western blot was performed by treating with 100 nM insulin for 20 minutes. Phosphorylation of AKT was inhibited in sHLO (FIG. 7I). Insulin responsiveness of PCK1, CREB 1, and FOXO1 was not suppressed in sHLO (FIGS. 7J-K). These results showed that PCK1 and other gluconeogenesis genes were not responsive to insulin in the sHLO. Furthermore, glucose production was not inhibited after insulin stimulation in sHLO (FIG. 7L).

### Example 5. Screening of Insulin Resistance and Fatty Liver Improving Drugs Using Fatty Liver Organoids

Since it was confirmed that fatty liver HLO showed insulin resistance, it was applied to the screening of drugs that improve NAFLD and insulin resistance. HLO were treated with fatty acids for 6 days to induce a fatty liver phenotype. Then, candidate drugs were exposed to the sHLO for 48 hours.

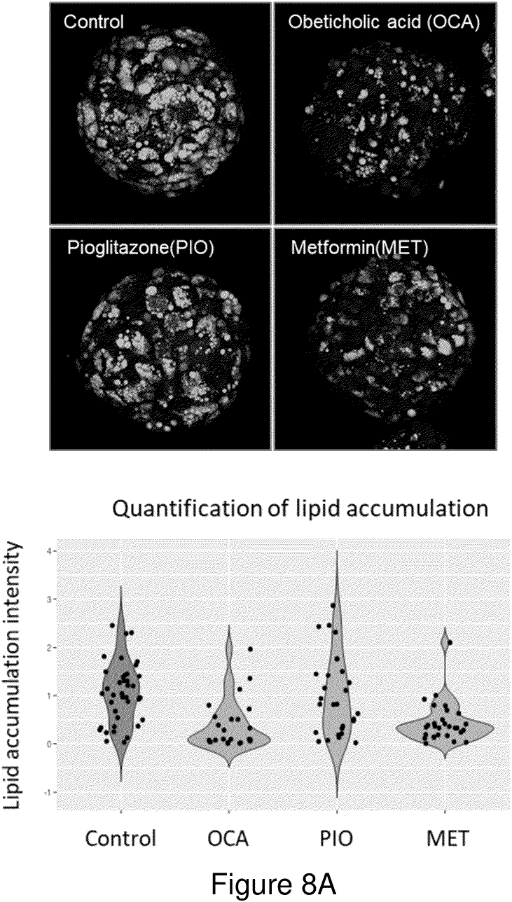

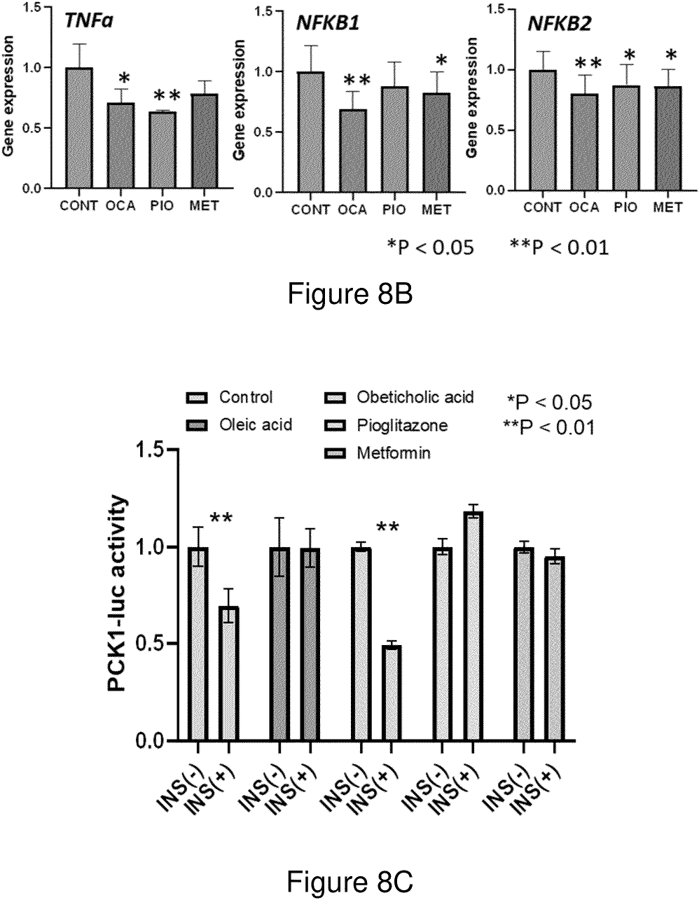

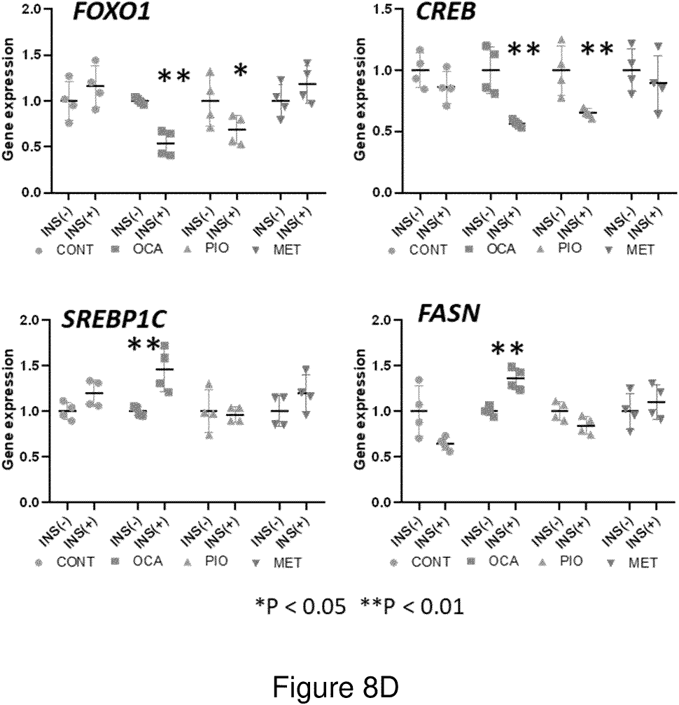

Obeticholic acid (OCA) treatment reduced fat accumulation and inflammatory gene expression (TNFa, NFKB1, NFKB2) (FIGS. 8A-B). Conversely, metformin (MET) treatment showed improvement in HLO fat accumulation but limited improvement in inflammatory response. Pioglitazone (PIO) treated HLOs showed limited improvement in fat accumulation and inflammatory response.

To investigate the improvement of insulin responsiveness in fatty liver HLO by treatment with candidate drugs, sHLO were incubated under insulin starvation for 24 hours and then exposed to insulin.

To analyze gluconeogenesis in response to insulin, luciferase activity was measured after treating HLO with 100 ng/mL insulin for 3 hours. Based on the PCK1-luciferase assay, OCA-treated fatty liver HLO showed improved insulin responsiveness (FIG. 8C).

To analyze expression of genes involved in insulin response, sHLO were treated with 100 ng/mL for 8 hours, and then RNA was extracted. OCA-treated sHLO had improved insulin responsiveness resulting in the suppression of gluconeogenesis regulatory genes and upregulation of lipogenic regulatory genes (FIG. 8D).

In summary, INSR and IRS2 expressed in the hepatic population of the multicomponent liver organoid, suggesting that the hepatic population of HLO responds to insulin signals and mimics the human insulin response. Human liver organoids showed hepatic insulin resistance when they accumulated fat and exhibited a fatty liver phenotype. In the fatty liver organoids, PCK1 was continuously activated and gluconeogenesis was enhanced. OCA treatment improved fat accumulation, inflammatory response, and gluconeogenesis/lipogenesis in fatty liver HLO. This suggests that drugs beneficial towards fat accumulation may also have the potential to improve hepatic insulin responsiveness. The human liver organoid model disclosed herein offers the opportunity to investigate hepatic insulin resistance and fat metabolism separated from other peripheral tissue metabolism found in living organisms.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “ a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed herein. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

What is claimed is:

1. A liver organoid comprising an insulin resistance reporter, wherein the insulin resistance reporter is operatively linked to an insulin-dependent gene of the liver organoid.

2. The liver organoid of claim 1, wherein the insulin-dependent gene is a gluconeogenesis gene or a lipogenesis gene.

3. The liver organoid of claim 1 or 2, wherein the insulin-dependent gene is selected from the group consisting of PCK1, G6PC, G6PC2, G6PC3, GSK3A, GSK3B, MTOR, GCK, FOXO1, CREB1, TFE1, TFE3, SREBP1C, FASN, ACLY, and ACC.

4. The liver organoid of any one of claims 1-3, wherein the insulin-dependent gene is PCK1.

5. The liver organoid of any one of claims 1-4, wherein expression of the insulin-dependent gene results in expression of the insulin resistance reporter.

6. The liver organoid of any one of claims 1-5, wherein the insulin-dependent gene and the insulin resistance reporter are separated by a bicistronic element.

7. The liver organoid of claim 6, wherein the bicistronic element is a self-cleaving peptide or an IRES.

8. The liver organoid of claim 7, wherein the self-cleaving peptide is a P2A, T2A, E2A, or F2A self-cleaving peptide.

9. The liver organoid of any one of claims 1-8, wherein the insulin resistance reporter has been integrated at the locus of the insulin-dependent gene using a CRISPR nuclease.

10. The liver organoid of claim 9, wherein the CRISPR nuclease is Cas9.

11. The liver organoid of any one of claims 1-10, wherein the insulin resistance reporter comprises one or more reporter genes.

12. The liver organoid of claim 11, wherein the one or more reporter genes comprise a gene encoding for a fluorescent protein or a gene encoding for a luminescent protein, or both.

13. The liver organoid of claim 12, wherein the fluorescent protein comprises mScarlet or the luminescent protein comprises luciferase.

14. The liver organoid of any one of claims 11-13, wherein the one or more reporter genes further comprise a resistance marker.

15. The liver organoid of claim 1-14, wherein the insulin resistance reporter comprises two or more reporter genes, and the two or more reporter genes are separated by one or more bicistronic elements.

16. The liver organoid of claim 15, wherein the one or more bicistronic elements comprise one or more self-cleaving peptides or IRES.

17. The liver organoid of claim 16, wherein the one or more self-cleaving peptides comprise a P2A, T2A, E2A, or F2A self-cleaving peptide.

18. The liver organoid of any one of claims 1-17, wherein the liver organoid is a fatty liver organoid or steatohepatitis liver organoid.

19. The liver organoid of claim 18, wherein the fatty liver organoid or steatohepatitis liver organoid comprises a large number of fat droplets compared to a normal liver organoid.

20. The liver organoid of claim 18 or 19, wherein the fatty liver organoid or steatohepatitis liver organoid is generated by contacting the liver organoid with fatty acids.

21. The liver organoid of claim 20, wherein the fatty acids comprise oleic acid, linoleic acid, palmitic acid, or stearic acid, or any combination thereof.

22. The liver organoid of any one of claims 18-21, wherein the fatty liver organoid or steatohepatitis liver organoid exhibits insulin resistance and/or a type 2 diabetic phenotype.

23. The liver organoid of claim 22, wherein insulin resistance comprises decreased AKT phosphorylation, reduced suppression of expression of PCK1, CREB1, or FOXO1 in response to insulin, or reduced suppression of gluconeogenesis in response to insulin, or any combination thereof, relative to a normal liver organoid.

24. The liver organoid of any one of claims 18-23, wherein the fatty liver organoid or steatohepatitis liver organoid exhibits more fat droplets, increased expression of DGAT½, or increased expression and/or secretion of pro-inflammatory cytokines, or any combination thereof, relative to a normal liver organoid.

25. The liver organoid of claim 24, wherein the pro-inflammatory cytokines comprise TNFa, TGFb, IL6, IL8, or IL1b, or any combination thereof.

26. The liver organoid of any one of claims 1-25, wherein the liver organoid is a mammalian or human liver organoid.

27. The liver organoid of any one of claims 1-26, wherein the liver organoid has been derived from pluripotent stem cells, induced pluripotent stem cells, or embryonic stem cells.

28. The liver organoid of any one of claims 1-27, wherein the liver organoid has been derived from cells from a subject having or at risk of developing a disease or disorder associated with insulin dysfunction.

29. The liver organoid of claim 28, wherein the disease or disorder associated with insulin dysfunction comprises diabetes, metabolic syndrome, fatty liver disease, steatohepatitis, obesity, cardiovascular disease, polycystic ovary syndrome, hyperglycemia, hyperinsulinemia, dyslipidemia, or any combination thereof.

30. The liver organoid of any one of claims 1-29, wherein the insulin resistance reporter comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 4.

31. A insulin responsive cell, tissue, or organoid comprising an insulin resistance reporter, wherein the insulin resistance reporter is operatively linked to an insulin-dependent gene of the insulin responsive cell, tissue, or organoid.

32. The insulin responsive cell, tissue, or organoid of claim 31, wherein the insulin-dependent gene is a gluconeogenesis gene or a lipogenesis gene.

33. The insulin responsive cell, tissue, or organoid of claim 31 or 32, wherein the insulin-dependent gene is selected from the group consisting of PCK1, G6PC, G6PC2, G6PC3, GSK3A, GSK3B, MTOR, GCK, FOXO1, CREB1, TFE1, TFE3, SREBP1C, FASN, ACLY, and ACC.

34. The insulin responsive cell, tissue, or organoid of any one of claims 31-33, wherein the insulin-dependent gene is PCK1.

35. The insulin responsive cell, tissue, or organoid of any one of claims 31-34, wherein expression of the insulin-dependent gene results in expression of the insulin resistance reporter.

36. The insulin responsive cell, tissue, or organoid of any one of claims 31-35, wherein the insulin-dependent gene and the insulin resistance reporter are separated by a bicistronic element.

37. The insulin responsive cell, tissue, or organoid of claim 36, wherein the bicistronic element is a self-cleaving peptide or an IRES.

38. The insulin responsive cell, tissue, or organoid of claim 37, wherein the self-cleaving peptide is a P2A, T2A, E2A, or F2A self-cleaving peptide.

39. The insulin responsive cell, tissue, or organoid of any one of claims 31-38, wherein the insulin resistance reporter has been integrated at the locus of the insulin-dependent gene using a CRISPR nuclease.

40. The insulin responsive cell, tissue, or organoid of claim 39, wherein the CRISPR nuclease is Cas9.

41. The insulin responsive cell, tissue, or organoid of any one of claims 31-40, wherein the insulin resistance reporter comprises one or more reporter genes.

42. The insulin responsive cell, tissue, or organoid of claim 41, wherein the one or more reporter genes comprise a gene encoding for a fluorescent protein or a gene encoding for a luminescent protein, or both.

43. The insulin responsive cell, tissue, or organoid of claim 42, wherein the fluorescent protein comprises mScarlet or the luminescent protein comprises luciferase.

44. The insulin responsive cell, tissue, or organoid of any one of claims 41-43, wherein the one or more reporter genes further comprise a resistance marker.

45. The insulin responsive cell, tissue, or organoid of any one of claim 31-44, wherein the insulin resistance reporter comprises two or more reporter genes, and the two or more reporter genes are separated by one or more bicistronic elements.

46. The insulin responsive cell, tissue, or organoid of claim 45, wherein the one or more bicistronic elements comprise one or more self-cleaving peptides or IRES.

47. The insulin responsive cell, tissue, or organoid of claim 46, wherein the one or more self-cleaving peptides comprise a P2A, T2A, E2A, or F2A self-cleaving peptide.

48. The insulin responsive cell, tissue, or organoid of any one of claims 31-47, wherein the insulin responsive cell, tissue, or organoid is a stem cell, induced pluripotent stem cell, embryonic stem cell, definitive endoderm cell, or a foregut cell.

49. The insulin responsive cell, tissue, or organoid of any one of claims 31-48, wherein the insulin resistance reporter comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 4.

50. An insulin resistance reporter, comprising one or more reporter genes flanked by a 5′ homology region and a 3′ homology region associated with an insulin-dependent gene.

51. The insulin resistance reporter of claim 50, wherein the insulin-dependent gene is a gluconeogenesis gene or a lipogenesis gene.

52. The insulin resistance reporter of claim 50 or 51, wherein the insulin-dependent gene is selected from the group consisting of PCK1, G6PC, G6PC2, G6PC3, GSK3A, GSK3B, MTOR, GCK, FOXO1, CREB1, TFE1, TFE3, SREBP1C, FASN, ACLY, and ACC.

53. The insulin resistance reporter of any one of claims 50-52, wherein the insulin-dependent gene is PCK1.

54. The insulin resistance reporter of any one of claims 50-53, wherein at least one of the one or more reporter genes and the 5′ homology region associated with an insulin-dependent gene are separated by a bicistronic element.

55. The insulin resistance reporter of claim 54, wherein the bicistronic element is a self-cleaving peptide or an IRES.

56. The insulin resistance reporter of claim 55, wherein the self-cleaving peptide is a P2A, T2A, E2A, or F2A self-cleaving peptide.

57. The insulin resistance reporter of any one of claims 50-56, wherein the one or more reporter genes comprise a gene encoding for a fluorescent protein or a gene encoding for a luminescent protein, or both.

58. The insulin resistance reporter of claim 57, wherein the fluorescent protein comprises mScarlet or the luminescent protein comprises luciferase.

59. The insulin resistance reporter of any one of claims 50-58, wherein the one or more reporter genes further comprise a resistance marker.

60. The insulin resistance reporter of any one of claims 50-59, wherein the insulin resistance reporter comprises two or more reporter genes, and the two or more reporter genes are separated by one or more bicistronic elements.

61. The insulin resistance reporter of claim 60, wherein the one or more bicistronic elements comprise one or more self-cleaving peptides or IRES.

62. The insulin resistance reporter of claim 61, wherein the one or more self-cleaving peptides comprise a P2A, T2A, E2A, or F2A self-cleaving peptide.

63. The insulin resistance reporter of any one of claims 50-62, wherein the 5′ homology region associated with the insulin-dependent gene comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% homology to SEQ ID NO: 6; and/or wherein the 3′ homology region associated with the insulin-dependent gene comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% homology to SEQ ID NO: 13.

64. The insulin resistance reporter of any one of claims 50-63, wherein the insulin resistance reporter comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 4.

65. An in vitro method of screening for candidate compounds for the treatment of a disease or disorder associated with insulin dysfunction, comprising:
contacting a liver organoid comprising an insulin resistance reporter or an insulin responsive cell, tissue, or organoid comprising an insulin resistance reporter with the candidate compounds; and
observing an improvement in the disease or disorder associated with insulin dysfunction in the liver organoid or the insulin responsive cell, tissue, or organoid.

66. The method of claim 65, wherein the disease or disorder associated with insulin dysfunction comprises diabetes, metabolic syndrome, fatty liver disease, steatohepatitis, obesity, cardiovascular disease, polycystic ovary syndrome, hyperglycemia, hyperinsulinemia, dyslipidemia, or any combination thereof.

67. The method of claim 65 or 66, wherein the liver organoid comprising the insulin resistance reporter is the liver organoid of any one of claims 1-30.

68. The method of any one of claims 65-67, wherein the insulin responsive cell, tissue, or organoid comprising the insulin resistance reporter is the insulin responsive cell, tissue, or organoid of any one of claims 31-49.

69. The method of any one of claims 65-68, wherein the liver organoid comprising the insulin resistance reporter is a fatty liver organoid or a steatohepatitis liver organoid.

70. The method of any one of claims 65-69, wherein observing an improvement in the disease or disorder associated with insulin dysfunction in the liver organoid comprises observing increased AKT phosphorylation, increased suppression of expression of PCK1, CREB1, or FOXO1 in response to insulin, or increased suppression of gluconeogenesis in response to insulin, or any combination thereof, relative to before the contacting step.

71. The method of any one of claims 65-70, wherein observing an improvement in the disease or disorder associated with insulin dysfunction in the liver organoid comprises observing a decreased number of fat droplets, reduced expression of DGAT½, or decreased expression and/or secretion of pro-inflammatory cytokines, or any combination thereof, relative to before the contacting step.

72. A method of monitoring insulin response in a subject, comprising:
transplanting a liver organoid comprising an insulin resistance reporter or an insulin responsive cell, tissue, or organoid comprising an insulin resistance reporter to the subject; and
monitoring expression of the insulin resistance reporter of the liver organoid or the insulin responsive cell, tissue, or organoid.

73. The method of claim 72, wherein the liver organoid comprising the insulin resistance reporter is the liver organoid of any one of claims 1-30.

74. The method of claim 72 or 73, wherein the insulin responsive cell, tissue, or organoid comprising the insulin resistance reporter is the insulin responsive cell, tissue, or organoid of any one of claims 31-49.

75. The method of any one of claims 72-74, wherein the subject has or is at risk of developing a disease or disorder associated with insulin dysfunction.

76. The liver organoid of any one of claims 1-30, the insulin responsive cell, tissue, or organoid of any one of claims 31-49, or the method of any one of claims 65-75, wherein the insulin resistance reporter is 3′ of the insulin-dependent gene, such that the insulin resistance reporter and the insulin-dependent gene are separated by a bicistronic element.

77. The liver organoid of any one of claims 1-30, the insulin responsive cell, tissue, or organoid of any one of claims 31-49, or the method of any one of claims 65-75, wherein the insulin resistance reporter is 5′ of the insulin-dependent gene, such that the insulin resistance reporter and the insulin-dependent gene are separated by a bicistronic element.
